(19) United States
(12) Patent Application Publication
(10) Pub. No.: US 2026/0369416 A1
(43) Pub. Date: Nov. 19, 2026

(54) ARTIFICIAL INTELLIGENCE BASED METHODS FOR DETERMINING A DISC-AT-RISK AND THE RISK FOR NAION

(71) Applicant: K2J Tech, Inc., Temple, TX (US)

(72) Inventors: Kyle SMITH, Temple, TX (US); Danny KIRCHMEIER, Belton, TX (US); Kerry SOLOMON, Sullivan's Island, SC (US); Jeremiah ELLIOTT, Temple, TX (US)

(73) Assignee: K2J Tech, Inc., Temple, TX (US)

(21) Appl. No.: 19/635,641

(22) Filed: Mar. 31, 2026

Related U.S. Application Data
(60) Provisional application No. 63/781,769, filed on Apr. 1, 2025.
(60) Provisional application No. 64/018,912, filed on Mar. 27, 2026.
(60) Provisional application No. 64/021,268, filed on Mar. 30, 2026.

(51) Int. Cl. G16H 50/30; G06T 7/00

(52) U.S. Cl. CPC G16H 50/30; G06T 7/0012; G06T 2207/20081; G06T 2207/30041

(57) ABSTRACT

The present disclosure provides methods for determining whether an eye has a disc-at-risk. The present disclosure provides methods for determining an increased risk of developing NAION in an eye of a patient. The methods use AI models to determine whether an eye has a disc-at-risk, and provide a notification that the eye has a disc-at-risk. The methods use AI models to determine an increased risk of developing NAION in an eye of a patient.

### CROSS-REFERENCE TO RELATED APPLICATION

This application claims the benefit of U.S. Provisional Application No. 63/781,769, filed Apr. 1, 2025; U.S. Provisional Application No. 64/018,912, filed Mar. 27, 2026; and U.S. Provisional Application No. 64/021,268, filed Mar. 30, 2026, each of which is incorporated by reference herein in its entirety.

### BACKGROUND OF THE DISCLOSURE

Nonarteritic anterior ischemic optic neuropathy (NAION) is the most common cause of acute optic nerve injury in individuals over 50 years old. NAION typically presents as an acute, monocular, painless, and irreversible loss of vision. Despite its frequency and severe impact on a patient's quality of life, many aspects of this disease, including its pathogenesis and effective treatments, remain unknown or unproven.

The risk factors for NAION include hypertension, diabetes mellitus, hyperlipidemia, arteriosclerosis, obstructive sleep apnea, smoking, being over 50 years of age, having a small optic cup-to-disc ratio (“disc-at-risk” or “crowded optic disc”), or optic disc drusen.

It has been reported that a statistical association exists between NAION and prescriptions for semaglutide medications like Ozempic, Wegovy, and Rybelsus. See e.g. Hathaway J T et al., “Risk of Nonarteritic Anterior Ischemic Optic Neuropathy in Patients Prescribed Semaglutide,” JAMA Ophthalmology, 142(8):732-739 (2024); Simonsen E et al., “Use of semaglutide and risk of non-arteritic anterior ischemic optic neuropathy: A Danish-Norwegian cohort study,” Diabetes Obesity Metabolism, 1-10 (2025). These studies suggest that NAION may be more common in patients who use a semaglutide for control of diabetes than in patients who received other diabetes medications, and conversely, that use of semaglutide may increase the risk of NAION.

While the risk of NAION is increased with semaglutide and related medications, the absolute risk remains low. Even so, given the dramatic increase in the use of such medications around the world, and the severe impact of the condition on a patient's quality of life, a cost-benefit analysis should be discussed between physicians prescribing semaglutide, and their patients. However, such analyses cannot be done without a complete assessment of the patient's risk factors for NAION.

Although a relationship between crowded optic discs and NAION is known, the pathophysiology is unclear and requires further studies. Furthermore, it is not understood why NAION is triggered in some eyes with crowded discs but not others. Hindering progress in this area of research is the lack of a uniform method for characterizing the morphology of the optic cup and disc. In most clinical scenarios, physicians examine the optic disc with slit lamp, direct, or indirect ophthalmoscopy and estimate the cup-to-disc ratio based on what they see. Sometimes they take a fundus photograph and manually measure the dimensions of the optic cup and disc. Although this subjective measurement technique may be more reliable than estimations made via ophthalmoscopy, the subjectivity of this technique results in variability in measurement across observers due to differences in their technique, training, and experience. An ocular coherence tomography (OCT) instrument can generate an objective and reasonably reliable assessment of the cup-to-disc ratio, but this is an expensive instrument unavailable to many clinicians. To this end, development of reliable and inexpensive methods that objectively characterize the anatomy of the optic cup and disc is beneficial.

### SUMMARY

The present disclosure provides a method for determining a disc-at-risk in a given eye of a given patient, the method comprising the steps: a. training a machine learning model with a plurality of eye fundus images from a plurality of patients; b. inputting an image of the given eye of the patient into the machine learning model; and c. outputting a status of disk-at-risk for the given patient and a corresponding confidence level.

The present disclosure provides a computer-implemented method for analyzing an image of a fundus for determining whether an eye has a disc-at-risk, the method comprising the steps: a. receiving the image of a fundus of an eye; b. detecting, using a first segmentation model, a perimeter of a cup; c. detecting, using a second segmentation model, a perimeter of a disc; d. calculating a physical characteristic of the optic disc from the detected perimeter of the cup and the detected perimeter of the disc; e. determining, based on the calculated physical characteristic of the optic disc and the image of the fundus as inputs, that the eye has a disc-at-risk; and f. providing a notification that the eye has a disc-at-risk.

The present disclosure provides computer-implemented method for analyzing an image of a fundus for determining whether an eye has a disc-at-risk, the method comprising the steps: a. receiving an image of a fundus of an eye; b. determining, using a first AI classification model, (i) a first status of whether the eye has a disc with minimal risk, and (ii) a first corresponding confidence level; c. determining, using a second AI classification model, (i) a second status of whether the eye has a disc with some risk, and (ii) a second corresponding confidence level; and d. determining, using a third AI classification model, (i) a third status of whether the eye has a disc with high risk, and (ii) a third corresponding confidence level.

The present disclosure provides a computer-implemented method for analyzing an image of a fundus for determining whether an eye has a disc-at-risk, the method comprising the steps: a. receiving an image of a fundus of an eye; b. determining, using an AI classification model, a risk status of the eye and a corresponding confidence level, wherein the risk status of the eye is selected from the group consisting of a disc with minimal risk, a disc with some risk, and a disc with high risk.

The present disclosure provides a computer-implemented method of determining an increased risk of developing NAION in an eye of a patient, the method comprising the steps: a. receiving the image of a fundus of the eye; b. detecting, using a first segmentation model, a perimeter of a cup; c. detecting, using a second segmentation model, a perimeter of a disc; d. calculating a physical characteristic of the optic disc from the detected perimeter of the cup and the detected perimeter of the disc; e. determining, based on the calculated physical characteristic of the optic disc and the image of the fundus as inputs, that the eye has an increased risk of developing NAION; and f. providing a notification that the eye has an increased risk of developing NAION.

The present disclosure provides a computer-implemented method of determining for determining an increased risk of developing NAION in an eye of a patient, the method comprising the steps: a. receiving an image of a fundus of the eye; b. determining, using a first AI classification model, (i) a first status of whether the eye has a disc with minimal risk, and (ii) a first corresponding confidence level; c. determining, using a second AI classification model, (i) a second status of whether the eye has a disc with some risk, and (ii) a second corresponding confidence level; d. determining, using a third AI classification model, (i) a third status of whether the eye has a disc with high risk, and (ii) a third corresponding confidence level; e. inputting, into an ensemble model, the first status of whether the eye has a disc with minimal risk, the first corresponding confidence level, the second status of whether the eye has a disc with some risk, the second corresponding confidence level, the third status of whether the eye has a disc with high risk, and the third corresponding confidence level, f. determining, using the ensemble model, (i) a fourth status of whether the eye has a disc-at-risk, and (ii) a fourth corresponding confidence level, and g. providing a notification that the eye has an increased risk of developing NAION based on the fourth status.

The present disclosure provides a computer-implemented method for determining whether an eye has an increased risk of developing NAION, the method comprising the steps: a. receiving an image of a fundus of an eye; b. determining, using an AI classification model, a risk status of the eye and a corresponding confidence level, wherein the risk status of the eye is selected from the group consisting of a disc with minimal risk, a disc with some risk, and a disc with high risk; and c. providing a notification that the eye has an increased risk of developing NAION when the risk status of the eye is a disc with high risk.

The present disclosure provides a program which, when executed by a processor, causes the processor to perform a method disclosed herein.

The present disclosure provides a computer-readable storage medium storing a program described herein.

The present disclosure provides a non-transitory computer-readable medium storing instructions that, when executed by a processor, causes the processor to perform one of the methods disclosed herein.

The present disclosure provides a non-transitory computer-readable storage medium comprising a report generated from performing the one of the methods disclosed herein.

The present disclosure provides an electronic device, comprising: one or more processors; a memory storing instructions that, when executed by the one or more processors, cause the one or more processors to perform one of the methods disclosed herein.

The present disclosure provides a system for analyzing an image of a fundus of an eye for determining whether the eye has a disc-at-risk, the system comprising: a. a camera capable of taking the image of the fundus of the eye; b. a processor communicatively coupled to the camera; and c. a non-transitory computer readable media communicatively coupled to the processor, and storing instructions that, when executed on the processor, causes the processor to perform the steps of: i. detecting, using a first segmentation model, a perimeter of a cup, wherein the first segmentation model is trained on cup segmentation data; ii. detecting, using a second segmentation model, a perimeter of a disc; iii. calculating a physical characteristic of the fundus from the detected perimeter of the cup and the detected perimeter of the disc; iv. determining, based on the calculated physical characteristic of the optic disc and the image of the fundus as inputs, that the eye has a disc-at risk; and v. providing a notification, via an output device, that the eye has a disc-at-risk.

The present disclosure provides a system for analyzing an image of a fundus of an eye for determining whether the eye has a disc-at-risk, the system comprising: a. a camera capable of taking the image of the fundus of the eye; b. a processor communicatively coupled to the camera; and c. a non-transitory computer readable media communicatively coupled to the processor, and storing instructions that, when executed on the processor, causes the processor to perform the steps of: i. receiving an image of a fundus of an eye; ii. determining, using a first AI classification model, (i) a first status of whether the eye has a disc with minimal risk, and (ii) a first corresponding confidence level; iii. determining, using a second AI classification model, (i) a second status of whether the eye has a disc with some risk, and (ii) a second corresponding confidence level; iv. determining, using a third AI classification model, (i) a third status of whether the eye has a disc with high risk, and (ii) a third corresponding confidence level; and v. providing a notification, via an output device, that the eye has a disc-at-risk if the third status of whether the eye has a disc with high risk is true.

The present disclosure provides a system for analyzing an image of a fundus of an eye for determining whether the eye has a disc-at-risk, the system comprising: a. a camera capable of taking an image of the fundus of the eye; b. a processor communicatively coupled to the camera; and c. a non-transitory computer readable media communicatively coupled to the processor, and storing instructions that, when executed on the processor, causes the processor to perform the steps of: i. receiving an image of a fundus of an eye; ii. determining, using an AI classification model, a risk status of the eye and a corresponding confidence level, wherein the risk status of the eye is selected from the group consisting of a disc with minimal risk, a disc with some risk, and a disc with high risk; and iii. providing a notification that the eye has a disc-at-risk when the risk status of the eye is a disc with high risk.

The present disclosure provides a system for analyzing an image of a fundus of an eye for determining whether the eye has an increased risk of developing NAION, the system comprising:
a. a camera capable of taking the image of the fundus of the eye;
b. a processor communicatively coupled to the camera; and
c. a non-transitory computer readable media communicatively coupled to the processor, and storing instructions that, when executed on the processor, causes the processor to perform the steps of:
   i. detecting, using a first segmentation model, a perimeter of a cup, wherein the first segmentation model is trained on cup segmentation data;
   ii. detecting, using a second segmentation model, a perimeter of a disc;
   iii. calculating a physical characteristic of the fundus from the detected perimeter of the cup and the detected perimeter of the disc;
   iv. determining, based on the calculated physical characteristic of the optic disc and the image of the fundus as inputs, that the eye has an increased risk of developing NAION; and
   v. providing a notification, via an output device, that the eye has an increased risk of developing NAION.

The present disclosure provides for analyzing an image of a fundus of an eye for determining whether the eye has an increased risk of developing NAION, the system comprising:
a. a camera capable of taking the image of the fundus of the eye;
b. a processor communicatively coupled to the camera; and
c. a non-transitory computer readable media communicatively coupled to the processor, and storing instructions that, when executed on the processor, causes the processor to perform the steps of:
   i. receiving an image of a fundus of an eye;
   ii. determining, using a first AI classification model, (i) a first status of whether the eye has a disc with minimal risk, and (ii) a first corresponding confidence level;
   iii. determining, using a second AI classification model, (i) a second status of whether the eye has a disc with some risk, and (ii) a second corresponding confidence level;
   iv. determining, using a third AI classification model, (i) a third status of whether the eye has a disc with high risk, and (ii) a third corresponding confidence level;
   v. determining, using the ensemble model, (i) a fourth status of whether the eye has a disc-at-risk, and (ii) a fourth corresponding confidence level; and
   vi. providing a notification, via an output device, that the eye has an increased risk of developing NAION based on the fourth status.

The present disclosure provides a system for analyzing an image of a fundus of an eye for determining whether the eye has an increased risk of developing NAION, the system comprising:
a. a camera capable of taking an image of the fundus of the eye;
b. a processor communicatively coupled to the camera; and
c. a non-transitory computer readable media communicatively coupled to the processor, and storing instructions that, when executed on the processor, causes the processor to perform the steps of:
   i. receiving an image of a fundus of an eye;
   ii. determining, using an AI classification model, a risk status of the eye and a corresponding confidence level, wherein the risk status of the eye is selected from the group consisting of a disc with minimal risk, a disc with some risk, and a disc with high risk; and
   iii. providing a notification that the eye has an increased risk of developing NAION when the risk status of the eye is a disc with high risk.

### BRIEF DESCRIPTION OF THE DRAWINGS

Aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIG. 1A illustrates an exemplary image of a fundus in a normal eye.

FIG. 1B illustrates the cup and the disc annotated in the image of a fundus in a normal eye.

FIG. 1C illustrates an exemplary image of a fundus in an eye with a disc-at-risk.

FIG. 2 illustrates a flow diagram of the steps of a method for determining a disc-at-risk in a given eye, in accordance with an aspect of the present disclosure.

FIG. 3 illustrates a flow diagram of the steps of a method for determining whether an eye has a disc-at-risk using the C/D model, in accordance with an aspect of the present disclosure.

FIG. 4 illustrates a flow diagram of the steps of a method for determining whether an eye has a disc-at-risk using the DAR model, in accordance with an aspect of the present disclosure.

FIG. 5 illustrates a flow diagram of the steps of a method for determining whether an eye has a disc-at-risk using the Color model, in accordance with an aspect of the present disclosure.

FIG. 6 illustrates a flow diagram of the steps of a method for determining whether an eye has a disc-at-risk using the Combined model, in accordance with an aspect of the present disclosure.

FIG. 7 illustrates schematically a computer control system or platform that is programmed or otherwise configured to implement methods provided herein, in accordance with an aspect of the present disclosure.

FIG. 8 illustrates a flow diagram of the steps of a method for analyzing an image of a fundus of an eye for determining whether an eye has a disc-at-risk using the C/D segmentation model, in accordance with an aspect of the present disclosure.

FIG. 9 illustrates a flow diagram of the steps of a method for analyzing an image of a fundus of an eye for determining whether an eye has a disc-at-risk using the Three-Binary Classification Model, in accordance with an aspect of the present disclosure.

FIG. 10 illustrates a flow diagram of the steps of a method for analyzing an image of a fundus of an eye for determining whether an eye has a disc-at-risk using the Ternary Classification Model, in accordance with an aspect of the present disclosure.

FIG. 11 illustrates the provider-estimated CDR of unaffected fellow eyes by medication subgroups. The graph illustrates the cumulative percentage (y-axis) of eyes that have a provider-estimated CDR less than a CDR value (x-axis).

FIG. 12 illustrates the cumulative distribution of photo-measured and OCT-measured CDR in unaffected fellow eyes vs control eyes. The graph plots the cumulative percentage (y-axis) of eyes that have a CDR less than a CDR value (x-axis).

### DETAILED DESCRIPTION

The present disclosure also describes various aspects of methods, apparatuses, and systems for processing of fundus images to derive anatomical parameters (e.g., cup perimeter, disc perimeter, color distribution), to support medical diagnosis and/or clinical decision.

The methods of the present disclosure advantageously provide automated methods to determine the C/D ratio of an eye in an objective and consistent manner. The measurements made are accurate and do not depend on the variability amongst the observers.

The present disclosure describes various aspects of computer-implemented methods, apparatuses, and systems for determining whether an eye has a disc-at-risk using artificial intelligence. The present disclosure describes various aspects of computer-implemented methods, apparatuses, and systems for determining whether an eye has an increased risk of developing NAION using artificial intelligence.

This description is not intended to be a detailed catalog of all the different ways in which the disclosure may be implemented, or all the features that may be added to the instant disclosure. For example, features illustrated with respect to one aspect may be incorporated into other aspect, and features illustrated with respect to a particular aspect may be deleted from that aspect. Thus, the disclosure contemplates that in some aspects of the disclosure, any feature or combination of features set forth herein can be excluded or omitted. In addition, numerous variations and additions to the various aspects suggested herein will be apparent to those skilled in the art in light of the instant disclosure, which do not depart from the instant disclosure. In other instances, well-known structures, interfaces, and processes have not been shown in detail in order not to unnecessarily obscure the disclosure.

It is intended that no part of this specification be construed to effect a disavowal of any part of the full scope of the disclosure. Hence, the following descriptions are intended to illustrate some particular aspects of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular aspects or aspects only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the disclosure described herein can be used in any combination. Moreover, the present disclosure also contemplates that in some aspects of the disclosure, any feature or combination of features set forth herein can be excluded or omitted.

The methods disclosed herein include and comprise one or more steps or actions for achieving the described method. The method steps and/or actions may be interchanged with one another without departing from the scope of the present disclosure. In other words, unless a specific order of steps or actions is required for proper operation of the aspect, the order and/or use of specific steps and/or actions may be modified without departing from the scope of the present disclosure.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a measurement value and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “exemplary” is used to mean serving as an example, instance, or illustration. Any embodiment or aspect described as “exemplary” is not necessarily to be construed as preferred or advantageous over other embodiments or aspects, nor is it meant to preclude equivalent structures and techniques known to those of ordinary skill in the art. Rather, use of the word exemplary is intended to present concepts in a concrete fashion, and the disclosed subject matter is not limited by such examples.

Reference will now be made in detail to the disclosed aspects, examples of which are illustrated in the accompanying drawings. Wherever convenient, the same reference numbers will be used throughout the drawings to refer to the same or like parts.

### Methods for Determining a Disc-at-Risk

The present disclosure provides methods for determining a disc-at-risk in a given eye of a given patient.

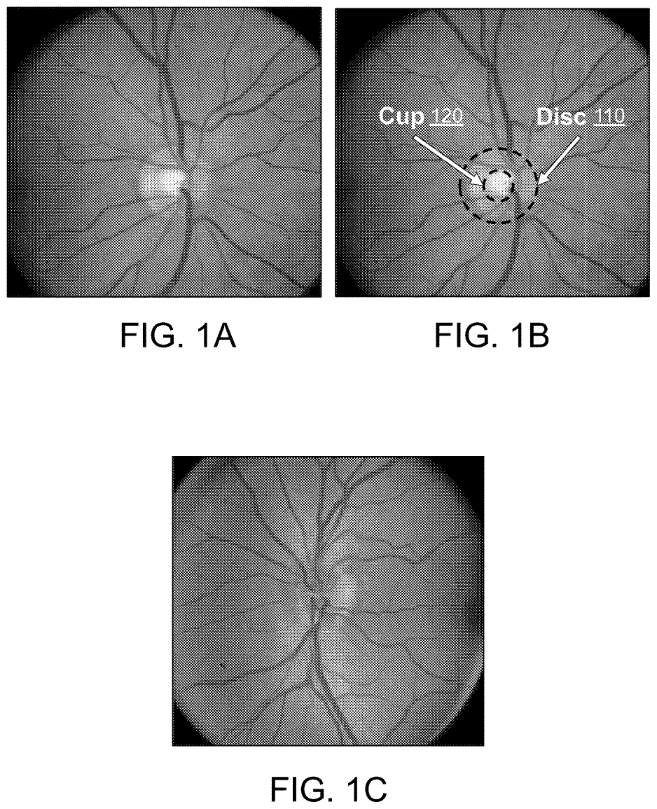

As used herein, “optic nerve head” or “optic disc” refers to the point of exit for ganglion cell axons leaving the eye. The optic disc represents the beginning of the optic nerve and is the point where the axons of retinal ganglion cells come together. A normal optic disc is orange to pink in color and may vary based on ethnicity (FIG. 1A). As used herein, the “optic cup” 120 is a white, cup-like area in the center of the optic disc. In the clinic, the inside back surface of the eye, referred to as “fundus of the eye” or “eye fundus,” can be visualized using an ophthalmoscope. Photographs of the retina and optic nerve can also be obtained with a fundus camera. The structures visible to an eye doctor using an ophthalmoscope include the retina, the optic disc 110, the optic cup 120, and blood vessels (FIG. 1).

The “cup-to-disc ratio” or C/D ratio compares the diameter of the optic cup to the diameter of the optic disc.

As used herein, “disc-at-risk” or “crowded disc” refers to an optic nerve head with a small diameter and a small cup-to-disc ratio, typically 0.2 or less (FIG. 1C). Without being bound by theory, a disc-at-risk is a risk factor for nonarteritic anterior ischemic optic neuropathy (NAION). In an aspect, a disc-at-risk has a C/D ratio of no more than about 0.05, no more than about 0.1, no more than about 0.15, no more than about 0.2, no more than about 0.25, no more than about 0.3, no more than about 0.35, no more than about 0.4, or no more than about 0.45. In an aspect, a disc-at-risk has a C/D ratio of no more than 0.2. In an aspect, a disc at minimal risk for NAION has a C/D ratio greater than about 0.25, greater than about 0.3, greater than about 0.35, greater than about 0.4, greater than about 0.45, greater than about 0.5, greater than about 0.55, greater than about 0.6, greater than about 0.65, greater than about 0.7, greater than about 0.75, or greater than about 0.8. In an aspect, a disc at some risk for NAION has a C/D ratio of about 0.2, about 0.25, about 0.3, about 0.35, about 0.4, about 0.45, or about 0.5. In an aspect, a disc at high risk for NAION has a C/D ratio of less than about 0.25, less than about 0.2, less than about 0.15, less than about 0.1, or less than about 0.05.

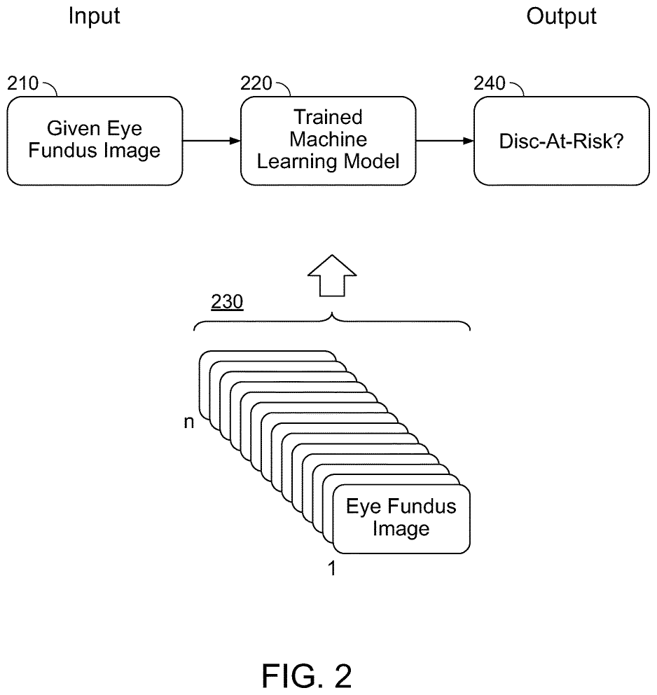

FIG. 2 is a schematic illustrating the overall steps of an aspect of the methods disclosed herein. The input to the method is a given fundus image of a given eye 210 in a given patient. In an aspect, the given fundus image is from a patient who has a disc-at-risk. In an aspect, the given fundus image is from a patient who has a normal disc. In an aspect, the given fundus image is from a patient who has a disc of unknown type. In an aspect, the given fundus image is taken by any technique known to one of skill in the art, including but not limited to an ophthalmoscope (e.g., direct, indirect, or confocal ophthalmoscope), a slit-lamp biomicroscope, or a fundus camera.

In this aspect, the given fundus image is received and inputted into a trained machine learning model 220. Then, the trained machine learning model determines whether the image of the given eye of the given patient has a disc-at-risk, and outputs the status 240. The machine learning model includes but is not limited to neural networks (e.g., deep neural networks) or other types of machine learning models, including non-linear models and/or linear models.

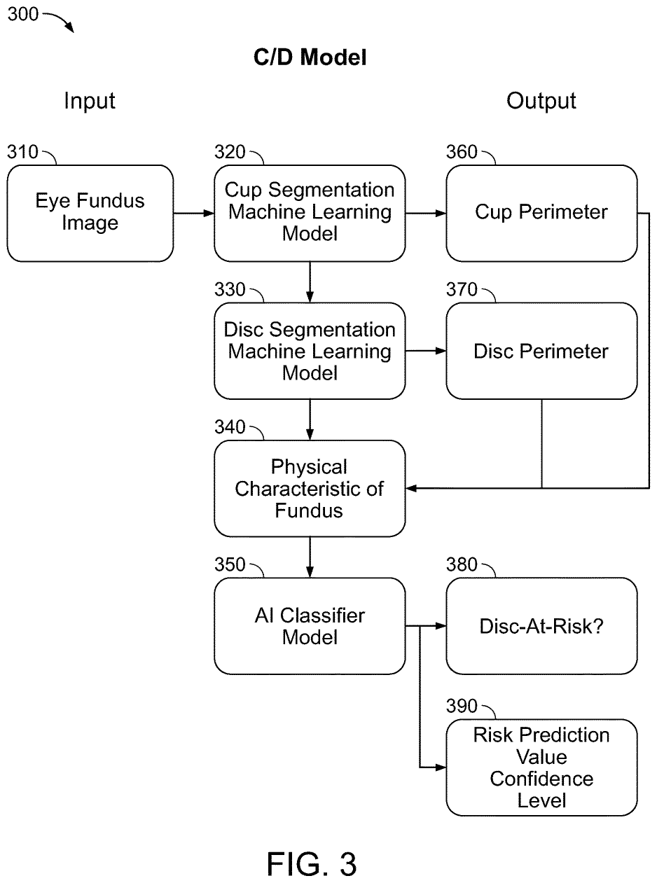

Neural networks can include feed-forward neural networks, recurrent neural networks (e.g., long short-term memory recurrent neural networks), convolutional neural networks or other forms of neural networks. Examples of trained machine learning models 220 are discussed with reference to FIGS. 3-6 and 8-10. In an aspect, the machine learning models described herein are convolutional neural networks.

In an aspect, the trained machine learning model 220 is trained with a plurality of eye fundus images 230 from a plurality of patients. The plurality of fundus images may be from any source. In an aspect, the plurality of fundus images encompasses eyes that are normal and eyes that have a disc-at-risk. In an aspect, the plurality of fundus images encompasses only eyes that are normal. In an aspect, the plurality of fundus images encompasses only eyes that have a disc-at-risk. The plurality of fundus images is taken by any technique known to one of skill in the art, including but not limited to an ophthalmoscope (e.g., direct, indirect, or confocal ophthalmoscope), a slit-lamp biomicroscope, or a fundus camera. In an aspect, the plurality of fundus images may include images taken by different techniques. In an aspect, the plurality of fundus images selected for training undergo preprocessing before being selected, including but not limited to image quality checks, smoothing, and scaling of brightness or contrast.

The machine learning model may be trained by any methods known to a person skilled in the art. For example, the training of the machine learning model may be unsupervised or supervised. In an aspect, a supervised training model trains on a plurality of eye fundus images from the plurality of patients that further comprises an associated status of disc-at-risk for each image. For example, each image used for training may be labeled as depicting a normal disc or a disc-at-risk, and the associated status (whether normal or disc-at-risk) is used as an input in the training. In an aspect, an unsupervised training model trains on unlabeled images.

In an aspect, the status 240 of a disk-at-risk for the given patient is whether the given image has a disc-at-risk or no disc-at-risk. In an aspect, the status of a disk-at-risk for the given patient is the likelihood that the given image has a disc-at-risk. In an aspect, the status of a disk-at-risk for the given patient is a binary value. In an aspect, the status of a disk-at-risk for the given patient is the likelihood that the given image has no disc-at-risk.

### Methods for Determining Whether an Eye has a Disc-at-Risk

The present disclosure provides methods and models for determining whether an eye has a disc-at-risk. FIGS. 3-6 and 8-10 are schematic illustrations of the overall steps of some aspects of the methods disclosed herein. In some aspects, the input to the methods is a fundus image of an eye (310, 410, 510, 610, 810, 910, or 1010). In an aspect, the fundus image is from a patient who has a disc-at-risk. In an aspect, the fundus image is from a patient who has a normal disc. In an aspect, the fundus image is from a patient who has a disc of unknown type. In an aspect, the fundus image is taken by any technique known to one of skill in the art, including but not limited to an ophthalmoscope (e.g., direct, indirect, or confocal ophthalmoscope), a slit-lamp biomicroscope, or a fundus camera.

In some aspects, the fundus image is received and inputted to one or more AI models for image segmentation. As used herein, “image segmentation” or “segmentation” refers to the process of partitioning a digital image into multiple image segments, also known as image regions or image objects (sets of pixels). The goal of segmentation is to simplify or change the representation of an image into something that is more meaningful and easier to analyze. Image segmentation is typically used to locate objects and boundaries (lines, curves, etc.) within images. More precisely, image segmentation is the process of assigning a label to every pixel in an image such that pixels with the same label share certain characteristics.

Some of the methods described herein utilize AI-based image segmentation techniques to locate a disc or a cup within the fundus image. Techniques and algorithms useful for image segmentation are generally known to a person of skill in the art. Examples of such segmentation methods include, but not limited to, thresholding (including Otsu's method and the maximum entropy method), k-means clustering, histogram-based methods, edge detection, and watershed transformation. Image segmentation can be performed using artificial intelligence techniques, such as machine learning and deep learning, which typically use neural networks with multiple layers.

In an aspect, a trained AI model detects a cup in the fundus image, and identifies a perimeter of the cup. In an aspect, an AI model is trained on cup segmentation data. For example, multiple images of fundus could be input into an AI model, with pixels associated with a cup labeled as such. In an aspect, an AI model training on cup segmentation data is supervised. In an aspect, an AI model training on cup segmentation data is unsupervised.

In an aspect, a trained AI model detects a disc in the fundus image, and identifies a perimeter of the disc. In an aspect, AI models is trained on disc segmentation data. For example, multiple images of fundus could be input into an AI model, with pixels associated with a disc labeled as such. In an aspect, AI models training on disc segmentation data is supervised. In an aspect, AI models training on disc segmentation data is unsupervised.

As used herein, “perimeter” refers to a continuous line forming the boundary of a closed geometric figure. Accordingly, the perimeter of a cup or a disc traces the outline of the cup or disk. In an aspect, the perimeter of a cup or a disc is the perimeter of a circle. In an aspect, the perimeter of a cup or a disc is the perimeter of an oval. In an aspect, the perimeter of a cup or a disc is the perimeter of an irregular shape. In an aspect, the perimeter of a cup or disc is smoothed. In an aspect, the perimeter of a cup or disc is the perimeter of a circle of best-fit. In an aspect, the perimeter of a cup or disc is the perimeter of an oval of best-fit.

In some aspects, information extracted from the images (e.g., by image segmentation) is inputted into AI classifier models together with the image of the fundus, to determine whether an eye has a disc-at-risk. As used herein, “AI classifier models” or “AI classification models” are any type of machine learning models that categorize data into predefined classes. Without being bound by theory, classifiers are a type of predictive modeling that learns class characteristics from input data and learns to assign possible classes to new data according to those learned characteristics.

AI classifier models may be based on supervised learning or unsupervised learning approaches. For example, the AI classifier models disclosed herein may be supervised, where each fundus image is labeled with an associated status, e.g., whether or not the image is one showing a disc-at-risk. In an aspect, an AI classifier model disclosed herein may be unsupervised, where each fundus image is unlabeled. The input data to an AI classifier model disclosed herein are fundus images, and can include any information associated with such images, including, but not limited to, information extracted from image segmentation of such images (e.g., perimeter, diameter, color, smoothness of edges). In some aspects, an AI classifier model uses as input the output from other AI models disclosed herein. For example, an AI classifier model disclosed herein could use image segmentation data (e.g., perimeter of cup and perimeter of disc) generated from a trained AI model disclosed herein, as input for classification of images. In another example, an AI classifier model disclosed herein could use the risk prediction value or associated confidence level output generated from a trained or classifier AI model disclosed herein, as input for classification of images.

Classification output may be discrete (binary or multiclass) or continuous. In some aspects, an AI classifier model disclosed herein outputs a discrete prediction. In an aspect, an AI classifier model disclosed herein classifies the fundus images into two, three, four, five, six, seven, eight, nine, ten, eleven, twelve, thirteen, fourteen, fifteen, sixteen, seventeen, eighteen, nineteen, or twenty classes. In an aspect, an AI classifier model is a binary model with two classes. In an aspect, an AI classifier model disclosed herein classifies the fundus images into two classes: minimal risk or risk. In an aspect, an AI classifier model disclosed herein is binary and classifies the fundus images into two classes: minimal risk or high risk. In an aspect, an AI classifier model is a ternary model with three classes. In an aspect, an AI classifier model disclosed herein classifies the fundus images into three classes: minimal risk, some risk, or high risk. In an aspect, an AI classifier model disclosed herein is ternary and classifies the fundus images into three classes: low risk, some risk, or high risk.

In some aspects, an AI classifier model disclosed herein outputs risk prediction values with an associated confidence level (continuous probabilities). As used herein, a “risk prediction value” refers to the calculated likelihood of a specific outcome or event occurring generated by a model and based on inputs. A risk prediction value is a unitless number, that can be expressed as a fractional or a whole number, and scaled accordingly to methods known to a person skilled in the art. For example, a risk prediction value of 0 and 1 may represent minimal risk and 100% risk respectively with fractional numbers between 0-1 representing the continuum of risk. In another example, a risk prediction value of 0, 1, and 2 may represent minimal risk, medium risk, and high risk respectively, in a scale that ranges from 0 to 3. In an aspect, a risk prediction value may be paired with a corresponding “confidence level,” which refers to a statistical measure of the percentage of results that can be expected to be within a specified range. For example, an AI classifier model could output a risk prediction value of zero (for example, minimal risk) with a confidence level or probability of 0.85. This means that the model believes that the risk of an image has an 85% chance of having no disc-at-risk and a 15% chance of having a disc-at-risk.

FIG. 3 is a schematic illustrating the overall steps of an aspect of the methods disclosed herein (C/D model 300). The input to the method is an image of a fundus of the eye of a patient 310. The fundus image is inputted into a first AI model 320, that has been trained on disc segmentation data. The first AI model detects a disc and a disc perimeter 360 in the image. The fundus image is also inputted into a second AI model 330, that has been trained on cup segmentation data. The second AI model detects a cup and a cup perimeter 370 in the image.

Next, a physical characteristic of the fundus is calculated from the detected cups and discs. Any physical characteristic that is associated with the image may be calculated, including, but not limited to, a vertical diameter of the cup, a vertical diameter of the disc, a horizontal diameter of the cup, a horizontal diameter of the disc, an oblique diameter of the cup, an oblique diameter of the disc, a ratio of the dimensions of the cup and the disc, a surface area of the cup and the disc, a ratio of the surface areas of the cup and the disc, a square-root of the ratio of the surface areas of the cup and the disc. In an aspect, a physical characteristic of the fundus is the cup-to-disc ratio. In an aspect, a physical characteristic of the fundus is the ratio of the surface area of the cup and the surface area of the disc. In an aspect, a physical characteristic of the fundus is the square root of the ratio of the surface area of the cup and the surface area of the disc.

The method further determines, using an AI classifier model 350 based on the calculated physical characteristic of the fundus 340 and the fundus image 310, whether the eye has a disc-at-risk or not 380. In an aspect, the method provides a notification that the eye has a disc-at-risk. In an aspect, AI classifier model 350 also generates a risk prediction value and a corresponding confidence level 390 that the eye has a disc-at-risk.

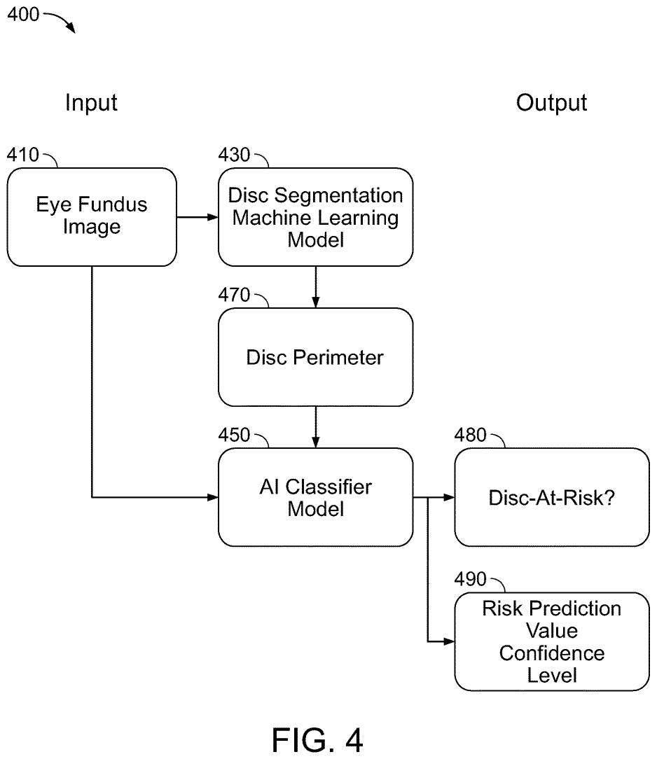

FIG. 4 is a schematic illustrating the overall steps of an aspect of the methods disclosed herein (disc-at-risk or DAR model 400). The input to the method is an image of a fundus of the eye of a patient 410. The fundus image is inputted into an AI model 420, that has been trained on disc segmentation data. The AI model detects a disc and a disc perimeter 460 in the image. The method further determines, using an AI classifier model 450 based on the detected perimeter of the disc 460 and the image of the fundus 410 as inputs, whether the eye has a disc-at-risk or not 480. In an aspect, the method provides a notification that the eye has a disc-at-risk. In an aspect, AI classifier model 450 also generates a risk prediction value and a corresponding confidence level 490 that the eye has a disc-at-risk.

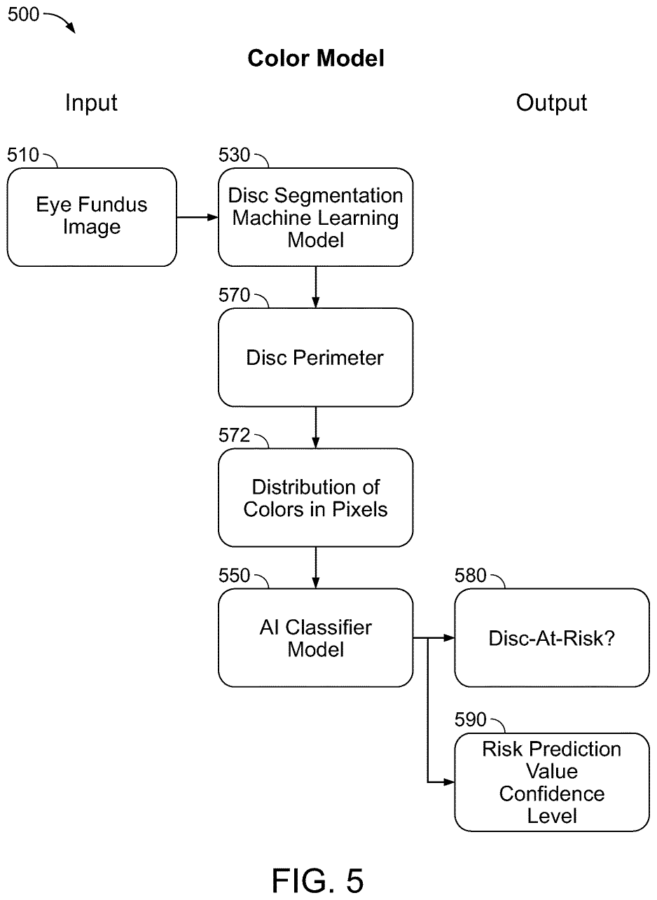

FIG. 5 is a schematic illustrating the overall steps of an aspect of the methods disclosed herein (Color model 500). The input to the method is an image of a fundus of the eye of a patient 510. The fundus image is inputted into an AI model 520, that has been trained on disc segmentation data. The AI model detects a disc and a disc perimeter 560 in the image. In an aspect, the method disclosed herein calculates the distribution of colors detected by the pixels 562 within the detected disc perimeter 560. As used herein, “color” of an object refers to the perception of a wavelength of light within the spectrum of visible light, that is determined by the scattering of light from the object. In an aspect, a camera that takes a color image of an object records the color of the object in each pixel of the image. Without being bound by theory, the color of an object provides information about the scattering properties of the object. In the case of a fundus, the color of the fundus image may provide information about the fundus, including but not limited to the depth of the cup, density of cells, perfusion in the retina, and health of the fundus. Visible light encompasses electromagnetic radiation with wavelengths of approximately 400 nm to 700 nm. In general, camera sensors “sense” color by separating detected light into bands of different wavelength, for example red, green, and blue. Since each perceived color can be expressed as a linear combination of the intensities of each of the bands, color can be detected at a single pixel by measuring the intensity detected of in each wavelength band. For example, in a red, green, blue (RGB) color model where each color is created by mixing varying intensities of red, green blue on a scale of 0 to 255, grey is made up of equal parts of red, green, and blue (128:128:128), while orange is made up of red, green, blue in the ratio of 230:159:0. In an aspect, color is encoded in the image pixel in red-green-blue (RGB) format. In an aspect, color is encoded in the image pixel in cyan-magenta-yellow-black (CMYK) format. In an aspect, the distribution of colors encoded in the pixels determined to be the disc provides additional information about the structure of the disc. In an aspect, the distribution of colors encoded in the pixels determined to be the disc provides additional information about whether the disc is a disc-at-risk. The method further determines, using an AI classifier model 550 based on the detected perimeter of the disc 560, the distribution of colors encoded in pixels 562 determined to be within the disc perimeter 560, and the image of the fundus 510 as inputs, whether the eye has a disc-at-risk or not 580. In an aspect, the method provides a notification that the eye has a disc-at-risk. In an aspect, AI classifier model 550 also generates a risk prediction value and a corresponding confidence level 590 that the eye has a disc-at-risk.

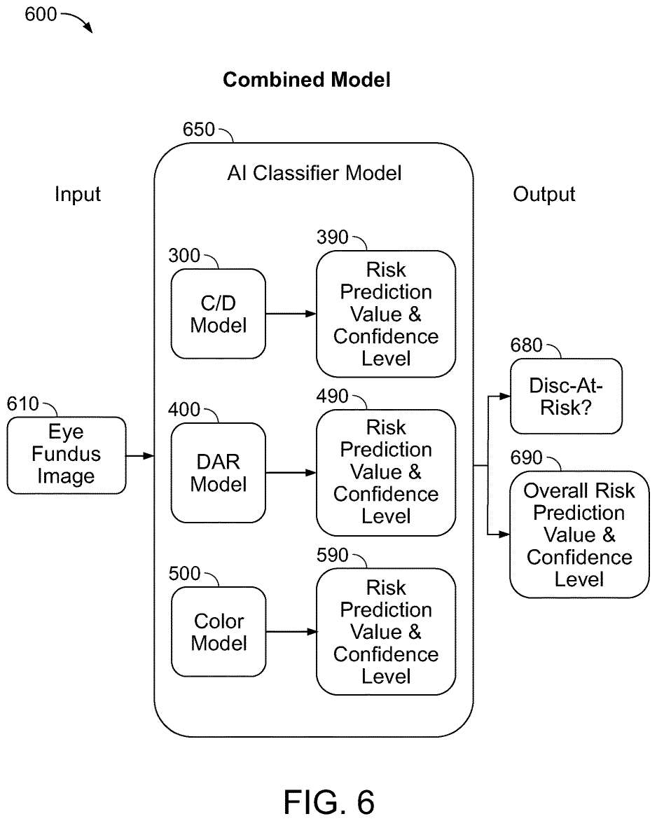

FIG. 6 is a schematic illustrating the overall steps of an aspect of the methods disclosed herein (Combined model 600). The input to the method is an image of a fundus of the eye of a patient 610. The fundus image is inputted into an AI classifier model 650, that is based on one or more of risk prediction values and one or more corresponding confidence levels. In an aspect, the AI classifier models receives one or more risk prediction values generated by one or more AI risk prediction models, selected from the group consisting of a C/D Model 300, a DAR Model 400, and a Color Model 500. The AI risk prediction models (300, 400, and 500) are described herein separately, as methods of determining whether an eye has a disc-at-risk (see FIGS. 3-5). The AI classifier model generates an overall risk prediction value and an overall confidence level 690 that the eye has a disc-at-risk 680, that is calculated from the one or more risk prediction values (390, 490, and 590) generated by the one or more AI risk prediction models selected from the group consisting of a C/D Model, a DAR Model, and a Color Model, weighted by the one or more corresponding confidence levels (390, 490, and 590).

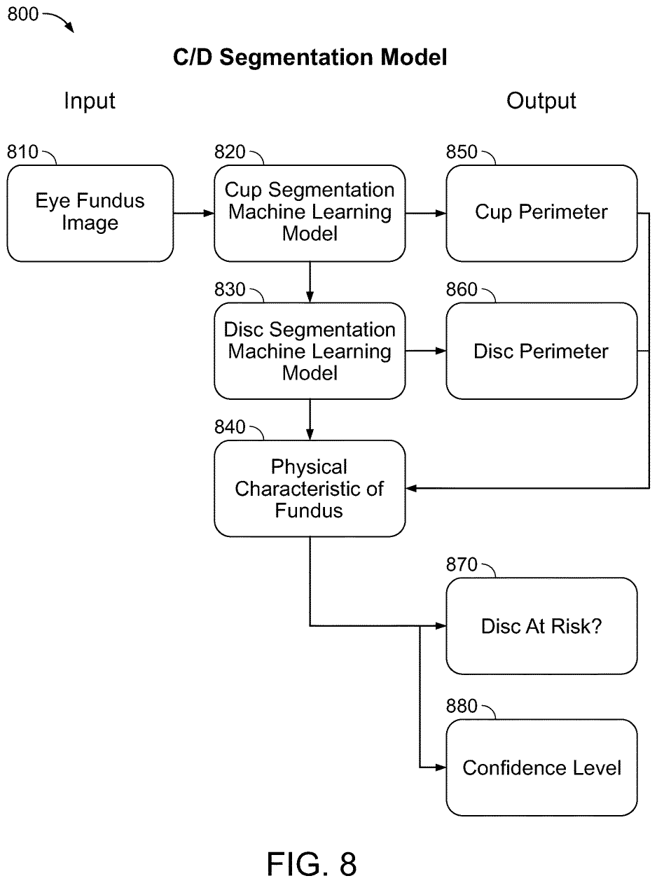

FIG. 8 is a schematic illustrating the overall steps of an aspect of the methods disclosed herein (C/D Segmentation Model 800). The input to the method is an image of a fundus of the eye of a patient 810. The fundus image is inputted into a cup segmentation machine learning model 820. The cup segmentation machine learning model 820 detects a cup and a cup perimeter 850 in the image. The fundus image is also inputted into a disc segmentation machine learning model 820. The disc segmentation machine learning model 820 detects a disc and a disc perimeter 860 in the image. Next, a physical characteristic of the fundus 840 is calculated from the segmented cup and disc. Any physical characteristic that is associated with the fundus image may be calculated, including, but not limited to, a vertical diameter of the cup, a vertical diameter of the disc, a horizontal diameter of the cup, a horizontal diameter of the disc, an oblique diameter of the cup, an oblique diameter of the disc, a ratio of the dimensions of the cup and the disc, a surface area of the cup and the disc, a ratio of the surface areas of the cup and the disc, a square-root of the ratio of the surface areas of the cup and the disc. In an aspect, a physical characteristic of the fundus is the cup-to-disc ratio. In an aspect, a physical characteristic of the fundus is the ratio of the surface area of the cup and the surface area of the disc. In an aspect, a physical characteristic of the fundus is the square root of the ratio of the surface area of the cup and the surface area of the disc. In an aspect, a physical characteristic of the fundus is the square root of the ratio of the diameter of the cup and the diameter of the disc.

The method further determines, based on the calculated physical characteristic of the optic disc 840 and the image of the fundus 810 as inputs, whether the eye has a disc-at-risk or not 870. In an aspect, the method provides a notification that the eye has a disc-at-risk. In an aspect, the method also generates a risk prediction value and a corresponding confidence level 880 that the eye has a disc-at-risk.

In an aspect, the cup segmentation machine learning model 820 is trained using a plurality of images of fundus where the perimeter of the cup in each image has been determined. In an aspect, the disc segmentation machine learning model 830 is trained using a plurality of images of fundus where the perimeter of the disc in each image has been determined.

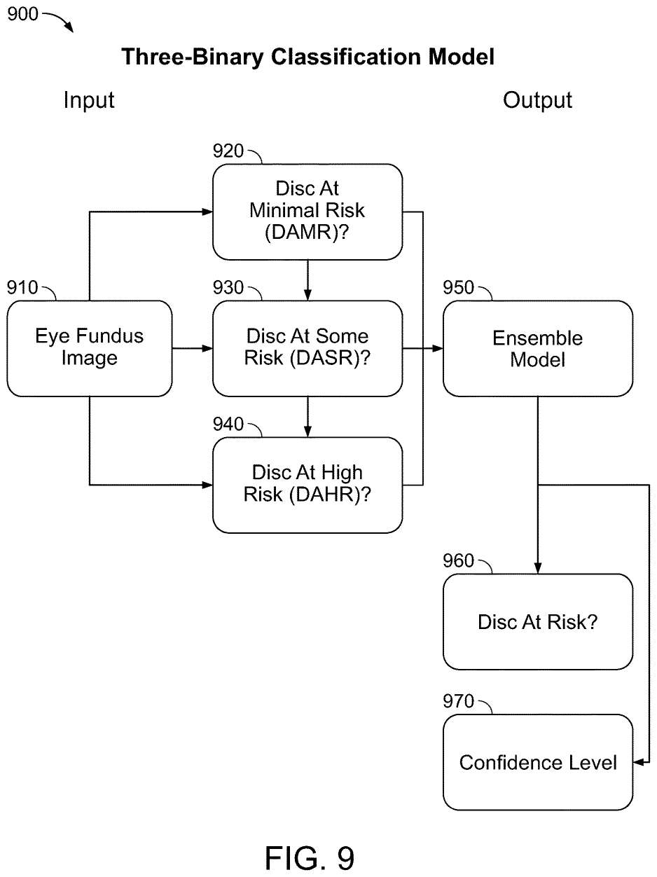

FIG. 9 is a schematic illustrating the overall steps of an aspect of the methods disclosed herein (Three-Binary Classification Model 900). In an aspect, the input to the method is an image of a fundus of the eye of a patient 910. In an aspect, the fundus image is inputted into a first AI classification model 920. In an aspect, the first AI classification model receives an image of a fundus of an eye and determines a first status of whether the eye has a disc with minimal risk. In an aspect, the first AI classification model receives an image of a fundus of an eye and determines a first status of whether the eye has a disc with minimal risk and a first corresponding confidence level. In an aspect, the fundus image is inputted into a second AI classification model 930. In an aspect, the second AI classification model receives an image of a fundus of an eye and determines a second status of whether the eye has a disc with some risk. In an aspect, the second AI classification model receives an image of a fundus of an eye and determines a second status of whether the eye has a disc with some risk and a second corresponding confidence level. In an aspect, the fundus image is inputted into a third AI classification model 940. In an aspect, the third AI classification model receives an image of a fundus of an eye and determines a third status of whether the eye has a disc with high risk. In an aspect, the third AI classification model receives an image of a fundus of an eye and determines a third status of whether the eye has a disc with high risk and a third corresponding confidence level.

The outputs of the AI classification models may be input into an ensemble model to determine whether the eye has a disc-at-risk. In an aspect, the method further comprises the steps of inputting, into an ensemble model 950, the first status of whether the eye has a disc with minimal risk, the first corresponding confidence level, the second status of whether the eye has a disc with some risk, the second corresponding confidence level, the third status of whether the eye has a disc with high risk, and the third corresponding confidence level. In an aspect, the method determines, using the ensemble model 950, a fourth status of whether the eye has a disc-at-risk 960, and a fourth corresponding confidence level 970. In an aspect, the fourth status of whether the eye has a disc-at-risk 960 is binary. In an aspect, the method provides a notification that the eye has a disc-at-risk based on the fourth status. In an aspect, the first status of whether the eye has a disc with minimal risk, the second status of whether the eye has a disc with some risk, and the third status of whether the eye has a disc with high risk are binary.

In an aspect, the first status of whether the eye has a disc with minimal risk, the second status of whether the eye has a disc with some risk, and the third status of whether the eye has a disc with high risk are binary. In an aspect, the first AI classification model 920 is trained with a plurality of images of fundus, wherein each image in the plurality of images has been assigned a status of whether the eye has a disc with minimal risk. In an aspect, the second AI classification mode 930 is trained with a plurality of images of fundus, wherein each image in the plurality of images has been assigned a status of whether the eye has a disc with some risk. In an aspect, the third AI classification model 940 is trained with a plurality of images of fundus, wherein each image in the plurality of images has been assigned a status of whether the eye has a disc with high risk.

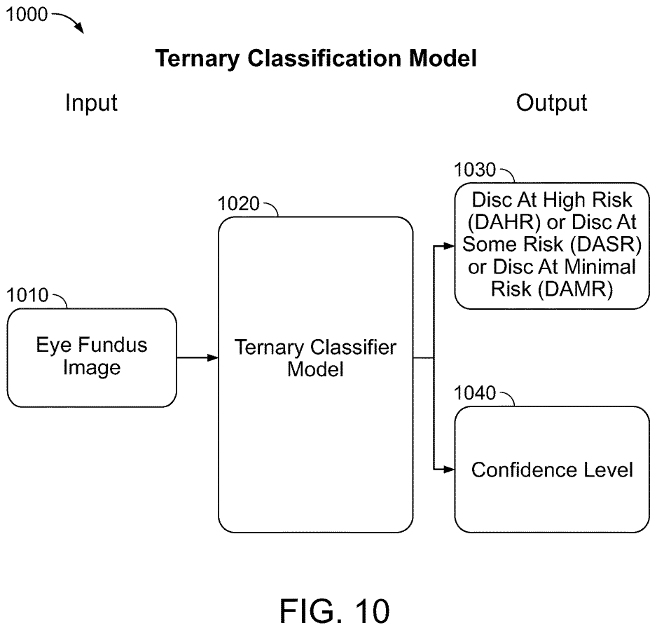

FIG. 10 is a schematic illustrating the overall steps of an aspect of the methods disclosed herein (Ternary Classification Model 1000). In an aspect, the input to the method is an image of a fundus of the eye of a patient 1010. In an aspect, the fundus image is inputted into an AI classification model 1020. In an aspect, the AI classification model 1020 receives an image of a fundus of an eye and determines a risk status of the eye. In an aspect, the AI classification model receives an image of a fundus of an eye 1010 and determines a risk status of the eye 1030 and a corresponding confidence level 1040. In an aspect, the first AI classification model receives an image of a fundus of an eye and determines a risk status of the eye. In an aspect, the risk status of the eye is selected from the group consisting of a disc with minimal risk, a disc with some risk, and a disc with high risk.

In an aspect, the AI classification model 1020 is trained with a plurality of images of fundus, wherein each image in the plurality of images has been assigned a status of whether the eye has a disc with minimal risk a disc with some risk, or a disc with high risk.

### Methods for Determining an Increased Risk of Developing NAION in an Eye of a Patient

The present disclosure provides methods for determining an increased risk of developing NAION in an eye of a patient, using a risk prediction value and a confidence level calculated by other methods described herein that are exemplified in FIGS. 3-6 and 8-10. While it is known that there is an association between NAION and a disc-at-risk, the reason and mechanism is unclear. Without being bound by theory, a disc-at-risk has an increased chance of developing NAION, in particular when semaglutide and related medications are administered. In some aspects, the input to the method is a fundus image of an eye (310, 410, 510, 610, 810, 910, or 1010). In an aspect, the method exemplified by FIG. 3, 4, 5, or 6, receives the image of a fundus of the eye (310, 410, 510, or 610 respectively) and calculates a risk prediction value and a confidence level (390, 490, 590, or 690) using an AI classifier model (350, 450, 550, or 650). In an aspect, the method exemplified by FIG. 8, 9, or 10, receives the image of a fundus of the eye (810, 910, or 1010 respectively) and determines whether the eye has a disc-at-risk (870, 960, or 1030) and a confidence level (880, 970, or 1040) using segmentation models (820 or 830) or classification models (920, 930, 940, 950, or 1020). In an aspect, the method exemplified by FIG. 9 determines whether the eye has a disc-at-risk 960 and a confidence level 970 using an ensemble model 950. In an aspect, the method determines that the patient has an increased risk of developing NAION when the eye has a disc-at-risk.

In an aspect, the method determines that the patient has an increased risk of developing NAION when the risk prediction value is greater than a first predetermined threshold. In an aspect, the method determines that the patient has minimal risk of developing NAION when the risk prediction value is less than a first predetermined threshold. It will be understood that the first predetermined threshold is not limited by design, but rather, one of ordinary skill in the art would be capable of choosing a predetermined threshold based on a given range of risk prediction values. In an aspect, a first predetermined threshold is about 0.1, about 0.2, about 0.3, about 0.4, about 0.5, about 0.6, about 0.7, about 0.8, or about 0.9 in a scale where the range of risk prediction values is from 0 to 1. In an aspect, a first predetermined threshold is at least 0.1, at least 0.2, at least 0.3, at least 0.4, at least 0.5, at least 0.6, at least 0.7, at least 0.8, or at least 0.9 in a scale where the range of risk prediction values is from 0 to 1.

In an aspect, a risk prediction value is binary. In an aspect, a risk prediction value is 0 or 1. For example, a binary risk prediction value of 0 and 1 may represent minimal risk and a finite risk respectively. In another example, a binary risk prediction value of 0 and 1 may represent some risk and certainty of occurring, respectively.

In an aspect, a method of determining an increased risk of developing NAION in an eye of a patient uses a C/D Segmentation Model described herein (e.g., FIG. 8). In an aspect, the input to the method is an image of a fundus of the eye of a patient 810. In an aspect, the fundus image is inputted into a cup segmentation machine learning model 820. In an aspect, the cup segmentation machine learning model 820 detects a cup and a cup perimeter 850 in the image. In an aspect, the fundus image is also inputted into a disc segmentation machine learning model 820. In an aspect, the disc segmentation machine learning model 820 detects a disc and a disc perimeter 860 in the image. Next, a physical characteristic of the fundus 840 is calculated from the segmented cup and disc. Any physical characteristic that is associated with the fundus image may be calculated, including, but not limited to, a vertical diameter of the cup, a vertical diameter of the disc, a horizontal diameter of the cup, a horizontal diameter of the disc, an oblique diameter of the cup, an oblique diameter of the disc, a ratio of the dimensions of the cup and the disc, a surface area of the cup and the disc, a ratio of the surface areas of the cup and the disc, a square-root of the ratio of the surface areas of the cup and the disc. In an aspect, a physical characteristic of the fundus is the cup-to-disc ratio. In an aspect, a physical characteristic of the fundus is the ratio of the surface area of the cup and the surface area of the disc. In an aspect, a physical characteristic of the fundus is the square root of the ratio of the surface area of the cup and the surface area of the disc. In an aspect, a physical characteristic of the fundus is the square root of the ratio of the diameter of the cup and the diameter of the disc.

In an aspect, the method further determines, based on the calculated physical characteristic of the optic disc 840 and the image of the fundus 810 as inputs, whether the eye has a disc-at-risk or not 870. In an aspect, the method provides a notification that the eye has an increased risk of developing NAION. In an aspect, the method also generates a risk prediction value and a corresponding confidence level 880 that the eye has a disc-at-risk. In an aspect, the calculated physical characteristic of the optic disc is the C/D ratio. In an aspect, the eye is determined to have an increased risk of developing NAION when the C/D ratio is less than or equal to about 0.1, less than or equal to about 0.11, less than or equal to about 0.12, less than or equal to about 0.13, less than or equal to about 0.14, less than or equal to about 0.15, less than or equal to about 0.16, less than or equal to about 0.17, less than or equal to about 0.18, less than or equal to about 0.19, less than or equal to about 0.2, less than or equal to about 0.21, less than or equal to about 0.22, less than or equal to about 0.23, less than or equal to about 0.24, less than or equal to about 0.25, less than or equal to about 0.26, less than or equal to about 0.27, less than or equal to about 0.28, less than or equal to about 0.29, or less than or equal to about 0.30. In an aspect, the eye is determined to have an increased risk of developing NAION when the C/D ratio is less than or equal to about 0.2.

In an aspect, the cup segmentation machine learning model 820 is trained using a plurality of images of fundus where the perimeter of the cup in each image has been determined. In an aspect, the disc segmentation machine learning model 830 is trained using a plurality of images of fundus where the perimeter of the disc in each image has been determined.

In an aspect, a method of determining an increased risk of developing NAION in an eye of a patient uses a Three-Binary Classification Model described herein (e.g., FIG. 9). In an aspect, the input to the method is an image of a fundus of the eye of a patient 910. In an aspect, the fundus image is inputted into a first AI classification model 920. In an aspect, the first AI classification model receives an image of a fundus of an eye and determines a first status of whether the eye has a disc with minimal risk. In an aspect, the first AI classification model receives an image of a fundus of an eye and determines a first status of whether the eye has a disc with minimal risk and a first corresponding confidence level. In an aspect, the fundus image is inputted into a second AI classification model 930. In an aspect, the second AI classification model receives an image of a fundus of an eye and determines a second status of whether the eye has a disc with some risk. In an aspect, the second AI classification model receives an image of a fundus of an eye and determines a second status of whether the eye has a disc with some risk and a second corresponding confidence level. In an aspect, the fundus image is inputted into a third AI classification model 940. In an aspect, the third AI classification model receives an image of a fundus of an eye and determines a third status of whether the eye has a disc with high risk. In an aspect, the third AI classification model receives an image of a fundus of an eye and determines a third status of whether the eye has a disc with high risk and a third corresponding confidence level.

In an aspect, an eye has a disc with minimal risk when the C/D ratio is greater than or equal to about 0.35, greater than or equal to about 0.4, greater than or equal to about 0.45, greater than or equal to about 0.5, greater than or equal to about 0.55, greater than or equal to about 0.6, greater than or equal to about 0.65, greater than or equal to about 0.7, greater than or equal to about 0.75, greater than or equal to about 0.8, greater than or equal to about 0.85, or greater than or equal to about 0.9. In an aspect, an eye has a disc with minimal risk when the C/D ratio is greater than or equal to about 0.4.

In an aspect, an eye has a disc with some risk when the C/D ratio is greater than or equal to about 0.15, and less than or equal to about 0.6. In an aspect, an eye has a disc with some risk when the C/D ratio is greater than or equal to about 0.15, and less than or equal to about 0.5. In an aspect, an eye has a disc with some risk when the C/D ratio is greater than or equal to about 0.15, and less than or equal to about 0.4. In an aspect, an eye has a disc with some risk when the C/D ratio is greater than or equal to about 0.2, and less than or equal to about 0.6. In an aspect, an eye has a disc with some risk when the C/D ratio is greater than or equal to about 0.2, and less than or equal to about 0.5. In an aspect, an eye has a disc with some risk when the C/D ratio is greater than or equal to about 0.2, and less than or equal to about 0.4. In an aspect, an eye has a disc with some risk when the C/D ratio is greater than or equal to about 0.25, and less than or equal to about 0.45. In an aspect, an eye has a disc with some risk when the C/D ratio is greater than or equal to about 0.2, and less than or equal to about 0.4.

In an aspect, an eye has a disc with high risk when the C/D ratio is less than or equal to about 0.1, less than or equal to about 0.11, less than or equal to about 0.12, less than or equal to about 0.13, less than or equal to about 0.14, less than or equal to about 0.15, less than or equal to about 0.16, less than or equal to about 0.17, less than or equal to about 0.18, less than or equal to about 0.19, less than or equal to about 0.2, less than or equal to about 0.21, less than or equal to about 0.22, less than or equal to about 0.23, less than or equal to about 0.24, less than or equal to about 0.25, less than or equal to about 0.26, less than or equal to about 0.27, less than or equal to about 0.28, less than or equal to about 0.29, or less than or equal to about 0.30. In an aspect, an eye has a disc with high risk when the C/D ratio is less than or equal to about 0.2.

The outputs of the AI classification models may be input into an ensemble model to determine whether the eye has a disc-at-risk. In an aspect, the method further comprises the steps of inputting, into an ensemble model 950, the first status of whether the eye has a disc with minimal risk, the first corresponding confidence level, the second status of whether the eye has a disc with some risk, the second corresponding confidence level, the third status of whether the eye has a disc with high risk, and the third corresponding confidence level. In an aspect, the method determines, using the ensemble model 950, a fourth status of whether the eye has a disc-at-risk 960, and a fourth corresponding confidence level 970. In an aspect, the fourth status of whether the eye has a disc-at-risk 960 is binary. In an aspect, the method provides a notification that the eye has an increased risk of developing NAION in the eye of a patient based on the fourth status. In an aspect, the first status of whether the eye has a disc with minimal risk, the second status of whether the eye has a disc with some risk, and the third status of whether the eye has a disc with high risk are binary.

In an aspect, the first AI classification model 920 is trained with a plurality of images of fundus, wherein each image in the plurality of images has been assigned a status of whether the eye has a disc with minimal risk. In an aspect, the second AI classification mode 930 is trained with a plurality of images of fundus, wherein each image in the plurality of images has been assigned a status of whether the eye has a disc with some risk. In an aspect, the third AI classification model 940 is trained with a plurality of images of fundus, wherein each image in the plurality of images has been assigned a status of whether the eye has a disc with high risk.

In an aspect, the C/D ratio of an eye having a disc-at-risk is less than or equal to about 0.1, less than or equal to about 0.11, less than or equal to about 0.12, less than or equal to about 013, less than or equal to about 0.14, less than or equal to about 0.15, less than or equal to about 0.16, less than or equal to about 0.17, less than or equal to about 0.18, less than or equal to about 0.19, less than or equal to about 0.2, less than or equal to about 0.21, less than or equal to about 0.22, less than or equal to about 0.23, less than or equal to about 0.24, less than or equal to about 0.25, less than or equal to about 0.26, less than or equal to about 0.27, less than or equal to about 0.28, less than or equal to about 0.29, less than or equal to about 0.3. In an aspect, the C/D ratio of an eye having a disc-at-risk is less than or equal to about 0.2.

In an aspect, a method of determining whether an eye has an increased risk of developing NAION uses a Ternary Classification Model described herein (e.g., FIG. 10). In an aspect, the input to the method is an image of a fundus of the eye of a patient 1010. In an aspect, the fundus image is inputted into an AI classification model 1020. In an aspect, the AI classification model 1020 receives an image of a fundus of an eye and determines a risk status of the eye. In an aspect, the AI classification model receives an image of a fundus of an eye 1010 and determines a risk status of the eye 1030 and a corresponding confidence level 1040. In an aspect, the first AI classification model receives an image of a fundus of an eye and determines a risk status of the eye. In an aspect, the risk status of the eye is selected from the group consisting of a disc with minimal risk, a disc with some risk, and a disc with high risk.

In an aspect, an eye has a risk status of a disc with minimal risk when the C/D ratio is greater than or equal to about 0.35, greater than or equal to about 0.4, greater than or equal to about 0.45, greater than or equal to about 0.5, greater than or equal to about 0.55, greater than or equal to about 0.6, greater than or equal to about 0.65, greater than or equal to about 0.7, greater than or equal to about 0.75, greater than or equal to about 0.8, greater than or equal to about 0.85, or greater than or equal to about 0.9. In an aspect, an eye has a risk status of disc with minimal risk when the C/D ratio is greater than or equal to about 0.4.

In an aspect, an eye has a risk status of a disc with some risk when the C/D ratio is greater than or equal to about 0.15, and less than or equal to about 0.6. In an aspect, an eye has a disc with some risk when the C/D ratio is greater than or equal to about 0.15, and less than or equal to about 0.5. In an aspect, an eye has a disc with some risk when the C/D ratio is greater than or equal to about 0.15, and less than or equal to about 0.4. In an aspect, an eye has a disc with some risk when the C/D ratio is greater than or equal to about 0.2, and less than or equal to about 0.6. In an aspect, an eye has a disc with some risk when the C/D ratio is greater than or equal to about 0.2, and less than or equal to about 0.5. In an aspect, an eye has a disc with some risk when the C/D ratio is greater than or equal to about 0.2, and less than or equal to about 0.4. In an aspect, an eye has a disc with some risk when the C/D ratio is greater than or equal to about 0.25, and less than or equal to about 0.45. In an aspect, an eye has a risk status of a disc with some risk when the C/D ratio is greater than or equal to about 0.2, and less than or equal to about 0.4.

In an aspect, an eye has risk status of a disc with high risk when the C/D ratio is less than or equal to about 0.1, less than or equal to about 0.11, less than or equal to about 0.12, less than or equal to about 0.13, less than or equal to about 0.14, less than or equal to about 0.15, less than or equal to about 0.16, less than or equal to about 0.17, less than or equal to about 0.18, less than or equal to about 0.19, less than or equal to about 0.2, less than or equal to about 0.21, less than or equal to about 0.22, less than or equal to about 0.23, less than or equal to about 0.24, less than or equal to about 0.25, less than or equal to about 0.26, less than or equal to about 0.27, less than or equal to about 0.28, less than or equal to about 0.29, or less than or equal to about 0.30. In an aspect, an eye has a risk status of a disc with high risk when the C/D ratio is less than or equal to about 0.2.

In an aspect, the AI classification model 1020 is trained with a plurality of images of fundus, wherein each image in the plurality of images has been assigned a status of whether the eye has a disc with minimal risk, a disc with some risk, or a disc with high risk.

The sensitivity and specificity of the AI classifier model may be tuned. In an aspect, an AI model or AI classifier is tuned such that during testing, it correctly identifies a predetermined percentage of eyes with labeled as having NAION. In an aspect, an AI classifier model is tuned such that a predetermined fraction of all patients have a risk prediction value of 1. In an aspect, an AI classifier model is tuned such that a predetermined fraction of all patients have a high risk of developing NAION. For example, an AI classifier model that calculates a risk prediction value and a confidence level may be calibrated such that a given proportion (e.g., 10%) of all input fundus images will be determined to have a high risk of developing NAION.

The methods described herein may also take into account additional information in determining the risk prediction value and the corresponding confidence level. For example, a method described herein may take patient data from electronic medical records (EMR) and determine a risk prediction value and a corresponding confidence level that is based in part on other risk factors for NAION like a patient's age and gender. In an aspect, a method of determining an increased risk of developing NAION in an eye of a patient further comprises: (a) receiving one or more risk information about the patient, the risk information selected from the group consisting of age, gender, blood pressure, and health history; (b) inputting the one or more risk information into the AI classifier model; and (c) generating the risk prediction value and the corresponding confidence level that the eye has a disc-at risk using the AI classifier model, wherein the AI classifier model includes the one or more risk information as an input. Risk information may be any information related to the patient, including but not limited to age, gender, blood pressure, health history, and future health actions (e.g., upcoming surgeries, prescribed medications not yet received). Such risk information may be incorporated by an AI classifier model when generating the risk prediction value and the corresponding confidence level that the eye has a disc-at risk, and improve the predictions made.

### Non-Transitory Computer-Readable Medium and Computer Systems

In some aspects of the present disclosure, software executing the instructions provided herein may be stored on a non-transitory computer-readable medium, wherein the software performs some or all of the steps of the present disclosure when executed on a processor. In some aspects of the present disclosure, a report generated from performing the methods disclosed herein may be stored on a non-transitory computer-readable medium.

Aspects of the present disclosure relate to algorithms executed in computer software. Though certain aspects may be described as written in particular programming languages, or executed on particular operating systems or computing platforms, it is understood that the system and method of the present disclosure is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present disclosure may be executed on any acceptable computing platform, including but not limited to a server, a cloud instance, a workstation, a thin client, a mobile device, an embedded microcontroller, a television, or any other suitable computing device known in the art.

Parts of this disclosure are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present disclosure, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this disclosure are described as communicatively coupled, or communicating over a variety of wireless or wired computer networks. For the purposes of this disclosure, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some aspects, elements of the networked portion of the disclosure may be implemented over a Virtual Private Network (VPN).

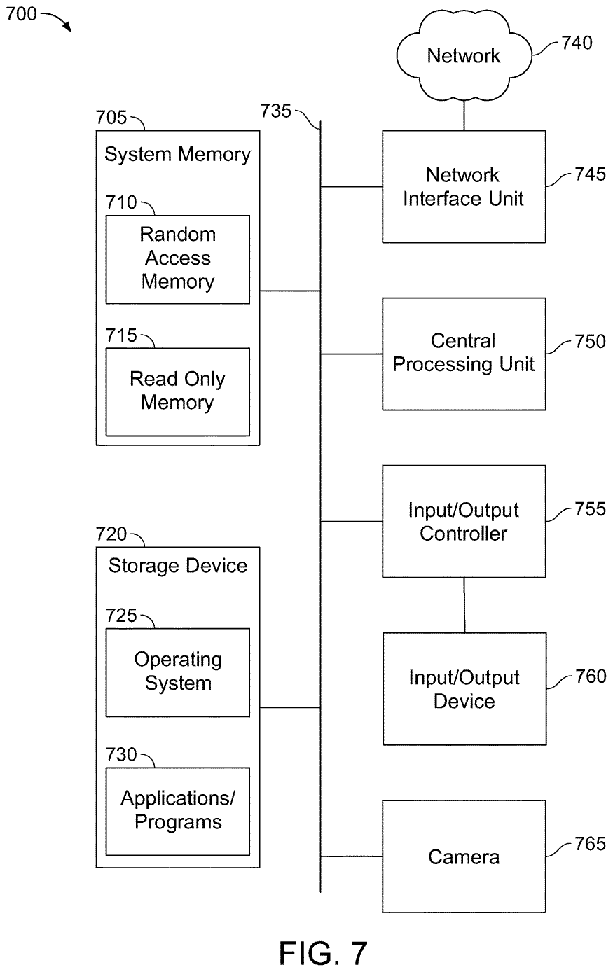

FIG. 7 and the following discussion are intended to provide a brief, general description of a suitable computing environment in which the disclosure may be implemented. While the disclosure is described above in the general context of program modules that execute in conjunction with an application program that runs on an operating system on a computer, those skilled in the art will recognize that the disclosure may also be implemented in combination with other program modules.

Generally, program modules include routines, programs, components, data structures, and other types of structures that perform particular tasks or implement particular abstract data types. Moreover, those skilled in the art will appreciate that the disclosure may be practiced with other computer system configurations, including hand-held devices, multiprocessor systems, microprocessor-based or programmable consumer electronics, minicomputers, mainframe computers, and the like. The disclosure may also be practiced in distributed computing environments where tasks are performed by remote processing devices that are linked through a communications network. In a distributed computing environment, program modules may be located in both local and remote memory storage devices.

The present disclosure provides computer systems that are programmed to implement methods of the disclosure. FIG. 7 depicts an illustrative computer architecture for a computer system 700 that is programmed or otherwise configured to implement the trained algorithm for the methods described herein. The computer architecture shown in FIG. 7 illustrates a conventional personal computer, including a central processing unit 750 (“CPU”), a system memory 705, including a random-access memory 710 (“RAM”) and a read-only memory (“ROM”) 715, and a system bus 735 that couples the system memory 705 to the CPU 750. A basic input/output system containing the basic routines that help to transfer information between elements within the computer, such as during startup, is stored in the ROM 715. The computer system 700 further includes a storage device 720 for storing an operating system 725, application/program 730, and data.

The storage device 720 is connected to the CPU 750 through a storage controller (not shown) connected to the bus 735. The storage device 720 and its associated computer-readable media, provide non-volatile storage for the computer system 700. Although the description of computer-readable media contained herein refers to a storage device, such as a hard disk or CD-ROM drive, it should be appreciated by those skilled in the art that computer-readable media can be any available media that can be accessed by the computer system 700.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data.

Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various aspects of the disclosure, the computer system 700 may operate in a networked environment using logical connections to remote computers through a network 740, such as TCP/IP network such as the Internet or an intranet. The computer system 700 may connect to the network 740 through a network interface unit 745 connected to the bus 735. It should be appreciated that the network interface unit 745 may also be utilized to connect to other types of networks and remote computer systems.

The computer system 700 may also include an input/output controller 755 for receiving and processing input from a number of input/output devices 760, including a keyboard, a mouse, a touchscreen, a camera, a microphone, a controller, a joystick, or other type of input device. Similarly, the input/output controller 755 may provide output to a display screen, a printer, a speaker, or other type of output device. The computer system 700 can connect to the input/output device 760 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 720 and RAM 710 of the computer system 700, including an operating system 725 suitable for controlling the operation of a networked computer. The storage device 720 and RAM 710 may also store one or more applications/programs 730. In particular, the storage device 720 and RAM 710 may store an application/program 730 for providing a variety of functionalities to a user. For instance, the application/program 730 may comprise many types of programs such as a word processing application, a spreadsheet application, a desktop publishing application, a database application, a gaming application, internet browsing application, electronic mail application, messaging application, and the like. According to an aspect of the present disclosure, the application/program 730 comprises a multiple functionality software application for providing word processing functionality, slide presentation functionality, spreadsheet functionality, database functionality and the like.

The computer system 700 in some aspects can include a camera 765 for taking an image of a fundus, that is internal to the computer system 700. The camera can be any camera capable of imaging the fundus of the eye. The camera can be hand held or table mounted with a chin and forehead rest. The camera can be mydriatic, or non-mydriatic. Examples of non-mydriatic cameras known to persons skilled in the art include but are not limited to the Zeiss VISUSCOUT 100, Topcon SIGNAL, 4MD Medical RetinaVue 100, and the VOLK Eyer2.

The computer system 700 can include or be in communication with an output device 760, e.g., an electronic display that comprises a user interface (UI) for providing, for example, notifications that the eye has a disc-at-risk. Examples of UI's include, without limitation, a graphical user interface (GUI) and web-based user interface. In an aspect, a system for determining whether an eye has a disc-at-risk provides a notification, via an output device, that the eye has a disc-at-risk. In an aspect, a system for determining an increased risk of developing NAION in an eye of a patient provides a notification, via an output device, that the eye has an increased risk of developing NAION when the risk prediction value is greater than a first threshold and the confidence level is greater than a second threshold. In an aspect, a system for determining an increased risk of developing NAION in an eye of a patient provides a notification, via an output device, that the eye has minimal risk of developing NAION when the risk prediction value is less than a third threshold and the confidence level is less than a fourth threshold.

In an aspect, a system for determining whether an eye has a disc-at-risk from an image of a fundus of the eye comprises: (a) a camera capable of taking an image of the fundus of the eye; (b) a processor communicatively coupled to the camera; and (c) a non-transitory computer readable media communicatively coupled to the processor, and storing instructions that, when executed on the processor, causes the processor to perform the methods disclosed herein.

In an aspect, a system for determining an increased risk of developing NAION in an eye of a patient comprises: (a) a camera capable of taking an image of the fundus of the eye; (b) a processor communicatively coupled to the camera; and (c) a non-transitory computer readable media communicatively coupled to the processor, and storing instructions that, when executed on the processor, causes the processor to perform the methods disclosed herein.

### EXAMPLES

### Example 1

A set of 10,000 fundus images is selected for testing and AI model training. The frequency of fundus images with a disc-at-risk in this testing set is about 15%, similar to the frequency of a eyes having a disc-at-risk in the general population. Each fundus image used for training is labeled by a subject matter expert to determine whether the eye has a disc-at-risk based on a predetermined threshold of C/D<0.33. A set of 1,000 test fundus images are evaluated with the trained AI model described herein to determine if each image has a disc-at-risk. The test fundus images are not used in training the model. Each test image is also labeled by a subject matter expert to determine whether the eye has a disc-at-risk based on a predetermined threshold of C/D<0.33. The AI-evaluated result for each test image is compared with the labeled result to determine true positives (sensitivity), false positives, true negatives (specificity), and false negatives.

The method of identifying a disc-at-risk described here provides a sensitivity of 94.6% and a specificity of 91.2%.

### Example 2

### Training for the C/D Segmentation Model:

A set of 10,000 fundus images is selected for testing and AI model training, drawn from a database of fundus images that represent the general population. For each image used in training, the C/D ratio is determined algorithmically. Briefly, an examiner marks out points on the perimeters of the optic disc and cup. A computer algorithm extrapolates an ellipse or a perturbed ellipse from the points marked out by the examiner. The areas of the optic disc and cup are determined from the areas of the extrapolated ellipses. The C/D ratio is calculated as the square root of the area of the cup divided by the area of the optic disc. The set of fundus images and the corresponding C/D ratio for each image are used as input for supervised training of the C/D Segmentation Model. A set of 1,000 test fundus images are evaluated with the trained C/D Segmentation Model. The test fundus images are not used in training the model. The trained C/D Segmentation Model takes in a test image and outputs the C/D ratio of the test eye. The accuracy of the model is tested by comparing the C/D ratio identified by the trained C/D Segmentation Model in a test image with that algorithmically as described here.

### Example 3

### Training for the Three-Binary Classification Model:

A set of 10,000 fundus images is selected for testing and AI model training, drawn from a database of fundus images that represent the general population. For each image used in training, the C/D ratio and the status of the eye (Disc at Minimal Risk, Disc a Some Risk, or Disc at High Risk) based on the C/D ratio are determined algorithmically. Briefly, an examiner marks out points on the perimeters of the optic disc and cup. A computer algorithm extrapolates an ellipse or a perturbed ellipse from the points marked out by the examiner. The areas of the optic disc and cup are determined from the areas of the extrapolated ellipses. The C/D ratio is calculated as the square root of the area of the cup divided by the area of the optic disc. The set of images and the corresponding status of the eye is used as input to train three separate status classification models using a convolutional neural network. Each classification model (Disc at Minimal Risk, Disc a Some Risk, or Disc at High Risk) is trained to take as input a test image and provide as output a binary (yes/no) determination of whether the optic disc in the test image has the corresponding status of the eye, and the confidence level of that determination (FIG. 9). For example, the Disc at Minimal Risk model is trained to take as input a test image and provide as output a yes/no determination of whether the optic disc in the test image has a disc at minimal risk, and the corresponding confidence level of that determination.

An ensemble model is also trained to take as input the binary (yes/no) determination of whether the optic disc in the test image is a disc that fits each category and the corresponding confidence level, and produce as output a binary determination of whether the optic disc is a disc-at-risk.

### Example 4

The Three-Binary Classification Model (FIG. 9) was trained and tested with a set of 1,000 test fundus images. The test fundus images are not used in training the model. The Disc At Minimal Risk classification model identified the images correctly with a sensitivity of 81% and a specificity of 94%. The Disc At Some Risk classification model identified the images correctly with a sensitivity of 77% and a specificity of 82%. The Disc At High Risk classification model identified the images correctly with a sensitivity of 86% and a specificity of 97%.

### Example 5

### Training for the Ternary Classification Model:

A set of 1,000 fundus images is selected for testing and AI model training, drawn from a database of fundus images that represent the general population. For each image used in training, the C/D ratio and the status of the eye (Disc at Minimal Risk, Disc a Some Risk, or Disc at High Risk) based on the C/D ratio are determined algorithmically. Briefly, an examiner marks out points on the perimeters of the optic disc and cup. A computer algorithm extrapolates an ellipse or a perturbed ellipse from the points marked out by the examiner. The areas of the optic disc and cup are determined from the areas of the extrapolated ellipses. The C/D ratio is calculated as the square root of the area of the cup divided by the area of the optic disc. The set of images and the corresponding status of the eye is used as input to train a classification model that has three classes (Disc at Minimal Risk, Disc a Some Risk, or Disc at High Risk). The classification model is trained to take as input a test image and provide as output a ternary determination (Disc at Minimal Risk, Disc a Some Risk, or Disc at High Risk) of whether the optic disc in the test image fits in one of the three classes, and the confidence level of that determination (FIG. 10). The test fundus images are not used in training the model.

### Example 6

To determine whether GLP-1 receptor agonist (GLP-1 RA) associated NAION has a high prevalence of crowded optic discs, a retrospective, multi-center, case control study of NAION cases was performed.

### Methods

Fundus photographs were taken from patients diagnosed with acute NAION over a 7-year period. Patients were excluded if there was reasonable concern for giant cell arteritis (GCA) or if the diagnosis was otherwise uncertain. Patients were also excluded if their list of medications, including GLP-1 RA drugs, was unavailable. This list was recorded during the first neuro-ophthalmology evaluation after the NAION event. Additional data collected from each patient includes the patient's age, sex, ethnicity, history of hypertension or diabetes, laterality of NAION, and provider estimated (cup-to-disc ratio) CDR of both eyes. Digital fundus photographs and ocular coherence tomography (OCT) retinal nerve fiber layer reports were included if available.

A preliminary control group of 2,000 fundus photographs representing the general population was obtained. The deidentified images were randomly selected from the Primary Eye Care version 2.0 dataset (IDHea PEC Datasheet: https://www.medrxiv.org/content/10.1101/2025.04.29.25326706v3.full-text) consisting of 1,117,868 images from 367,679 subjects from 64 optometry practices across the USA and Australia. After eliminating images from patients without photographs of both eyes and a corresponding OCT, and after sex matching to the NAION group, the final control group consisted of 930 fundus photographs. These photographs included 465 image pairs (i.e., right and left eye from the same patient).

The cup-to-disc ratio (CDR) of each fellow eye and control eye fundus photograph was measured independently by the three examiners using imaging software that allowed annotation directly on the image. These annotations generated one ellipse for the disc perimeter and another for the cup. The software calculated the area of each ellipse. The CDR was calculated by taking the square root of the cup area divided by the square root of the disc area. The average of the cup-to-disc ratios defined by the three examiners was used in the analysis.

Fundus photographs of the NAION eyes were used to verify the diagnosis but were not used for evaluation of CDR because disc swelling in the acute phase of NAION renders CDR evaluation unreliable. Likewise, CDR evaluation of late stage NAION with optic atrophy is also unreliable due to progressive volume loss and thinning of the neural rim that may occur. For this reason, CDR measurements from photographs were performed only on unaffected fellow eyes of patients with documented unilateral NAION. Bilateral NAION cases were not included in the optic disc analysis nor in the comparisons with the control group.

Patient demographics and NAION characteristics were evaluated using descriptive and Chi-square statistics. For age and CDR comparisons, a two-tailed Student's t-test was used. Odds ratios were calculated with a 95% confidence interval, and p values less than 0.05 were considered statistically significant.

### Results

Table 1 summarizes the characteristics of 2,169 patients with NAION included in the analysis. NAION patients with no prior GLP-1 RA use were older (No GLP group; n=1908; mean age, 62.9 years) than semaglutide users (Semaglutide group: n=167; mean age, 60.6 years) (p=0.005) and non-semaglutide GLP-1 RA users (Other GLP group: n=94; mean age, 59.8 years) (p=0.003). The non-semaglutide medications included dulaglutide (39), tirzepatide (26), liraglutide (24), exenatide (5).

Patients with NAION were more likely to be male (58.6%) than female (41.4%) (odds ratio (OR): 1.41 [1.25-1.59], p<0.001). Sex distribution did not differ significantly among the three groups (p=0.497). Unilateral NAION more frequently involved the right eye (54.2%) than the left (45.8%) (OR: 1.18 [1.04-1.35], p<0.001). Bilateral involvement was more common in semaglutide patients (29.9%) than in No GLP patients (15.5%) (OR: 2.33 [1.63-3.31], p<0.001). Patients on semaglutide without diabetes had no significant difference in bilaterality (OR: 1.69 [0.72-3.99], p=0.226). There was no difference in bilaterality between the Other GLP group (11.7% o) and the No GLP group (OR: 0.72 [0.38-1.37], p=0.317).

TABLE 1
Patient characteristics.

| | All Groups | | No GLP | | Semaglutide | | Other GLP | |
|---|---|---|---|---|---|---|---|---|
| Number | 2169 | | 1908 | (88%) | 167 | (8%) | 94 | (4%) |
| Age [Range] | 62.6 | [19-96] | 62.9 | [19-96] | 60.6 | [38-81] | 59.8 | [35-78] |

Sex

| | | | | | | | | |
|---|---|---|---|---|---|---|---|---|
| Male | 1270 | (59%) | 1126 | (59%) | 92 | (55%) | 52 | (55%) |
| Female | 899 | (41%) | 782 | (41%) | 75 | (45%) | 42 | (45%) |

Race/Ethnicity

| White/Caucasian | 1556 | (72%) | 1351 | (71%) | 135 | (81%) | 70 | (75%) |
|---|---|---|---|---|---|---|---|---|
| Black/African American | 71 | (3%) | 67 | (4%) | 4 | (2%) | 0 | |
| Hispanic | 44 | (2%) | 38 | (2%) | 3 | (2%) | 3 | (3%) |
| Asian/Pacific Islander | 11 | (1%) | 11 | (1%) | 0 | | 0 | |
| Other | 246 | (11%) | 223 | (12%) | 12 | (7%) | 11 | (12%) |
| Unknown | 241 | (11%) | 218 | (11%) | 13 | (8%) | 10 | (11%) |
| Diabetes mellitus | 793 | (37%) | 569 | (30%) | 137 | (82%) | 87 | (93%) |
| Hypertension | 1402 | (65%) | 1204 | (63%) | 122 | (73%) | 76 | (81%) |

Laterality of ION

| | | | | | | | | |
|---|---|---|---|---|---|---|---|---|
| Unilateral | 1812 | (84%) | 1612 | (84%) | 117 | (70%) | 83 | (88%) |
| Right Eye | 982 | (54%) | 884 | (55%) | 65 | (56%) | 33 | (40%) |
| Left Eye | 830 | (46%) | 728 | (45%) | 52 | (44%) | 50 | (60%) |
| Bilateral | 357 | (16%) | 296 | (16%) | 50 | (30%) | 11 | (12%) |

Table 2 summarizes the CDR analysis of the unaffected fellow eyes. The CDR of 1,812 unaffected fellow eyes was assessed with 3 methods: provider-estimated (1,668 eyes), photo-measured (235 eyes), and OCT-measured (654 eyes). The OCT-measured method included 591 scans performed with the Zeiss Cirrus (Carl Zeiss Meditec, Jena, Germany) and 63 performed with Topcon Maestro (Topcon Healthcare, Tokyo, Japan). None of the eyes were evaluated with both OCT devices.

TABLE 2
CDR of Unaffected Fellow Eyes - Various Assessment Methods

| | | | |
|---|---|---|---|
| All groups | No GLP | Semaglutide | Other GLP |

Provider Estimated

| Total Eyes | 1668 | | 1485 | | 107 | | 76 | |
|---|---|---|---|---|---|---|---|---|
| CDR ≤ 0.20 (%) | 1376 | (83%) | 1222 | (82%) | 91 | (85%) | 63 | (83%) |
| CDR ≤ 0.30 (%) | 1583 | (95%) | 1407 | (95%) | 103 | (96%) | 73 | (96%) |
| CDR ≤ 0.40 (%) | 1645 | (99%) | 1465 | (99%) | 106 | (99%) | 74 | (97%) |
| CDR ≤ 0.50 (%) | 1663 | (100%) | 1480 | (100%) | 107 | (100%) | 76 | (100%) |
| CDR range | 0.0-0.7 | | 0.0-0.7 | | 0.0-0.5 | | 0.0-0.5 | |
| Mean CDR | 0.13 | | 0.13 | [Ref] | 0.13 | [p = 0.539] | 0.12 | [p = 0.515] |
| Median CDR (SD) | 0.10 | (0.12) | 0.10 | (0.12) | 0.10 | (0.11) | 0.10 | (0.12) |

Photo Measured

| Number | 235 | | 206 | | 20 | | 9 | |
|---|---|---|---|---|---|---|---|---|
| CDR ≤ 0.20 (%) | 126 | (54%) | 111 | (54%) | 12 | (60%) | 3 | (33%) |
| CDR ≤0.30 (%) | 179 | (76%) | 162 | (79%) | 13 | (65%) | 4 | (44%) |
| CDR ≤ 0.40 (%) | 222 | (95%) | 196 | (95%) | 19 | (95%) | 7 | (78%) |
| CDR ≤ 0.50 (%) | 235 | (100%) | 206 | (100%) | 20 | (100%) | 9 | (100%) |
| CDR range | 0.01-0.48 | | 0.10-0.48 | | 0.01-0.40 | | 0.08-0.45 | |
| Mean CDR | 0.20 | | 0.20 | [Ref] | 0.20 | [p = 0.874] | 0.26 | [p = 0.144] |
| Median CDR (SD) | 0.19 | (0.12) | 0.19 | (0.11) | 0.18 | (0.13) | 0.30 | (0.14) |

OCT Measured

| Number | 654 | | 588 | | 40 | | 26 | |
|---|---|---|---|---|---|---|---|---|
| CDR ≤ 0.20 (%) | 341 | (52%) | 300 | (51%) | 26 | (65%) | 15 | (58%) |
| CDR ≤ 0.30 (%) | 412 | (63%) | 366 | (62%) | 29 | (73%) | 17 | (65%) |
| CDR ≤ 0.40 (%) | 500 | (77%) | 445 | (76%) | 35 | (88%) | 20 | (77%) |
| CDR ≤ 0.50 (%) | 567 | (87%) | 508 | (86%) | 36 | (90%) | 23 | (89%) |
| CDR ≤ 0.60 (%) | 617 | (94%) | 554 | (94%) | 39 | (98%) | 24 | (92%) |
| CDR range | 0.00-0.94 | | 0.00-0.94 | | 0.00-0.66 | | 0.00-0.79 | |
| Mean CDR | 0.25 | | 0.25 | [Ref] | 0.19 | [p = 0.075] | 0.24 | [p = 0.688] |
| Median CDR (SD) | 0.18 | (0.20) | 0.20 | (0.20) | 0.09 | (0.17) | 0.13 | (0.22) |

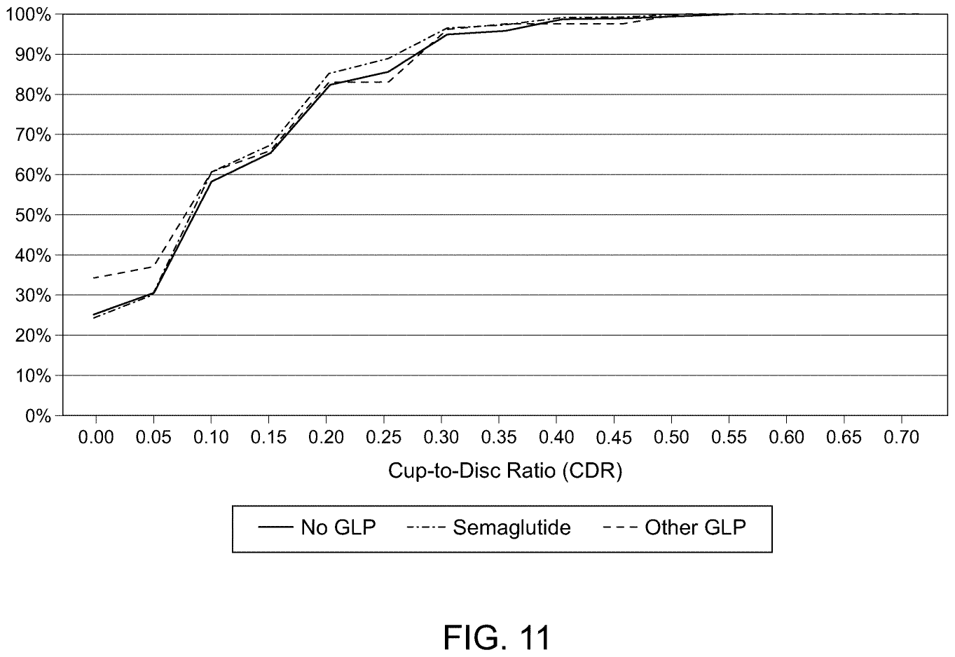

Using the provider-estimated method, as shown in FIG. 11, the mean and median provider-estimated CDR did not differ among the three groups (No GLP, Semaglutide, or Other GLP), averaging 0.13. Using the photo-measured CDR method, the average CDR of the measured fundus photos was 0.20 and did not demonstrate a CDR difference between groups. Using the OCT-measured CDR method, the average CDR in the Cirrus group (0.25) did not differ significantly from the Maestro group (0.23) (p=0.503), and there was no difference in the OCT-measured CDR between the three medication subgroups.

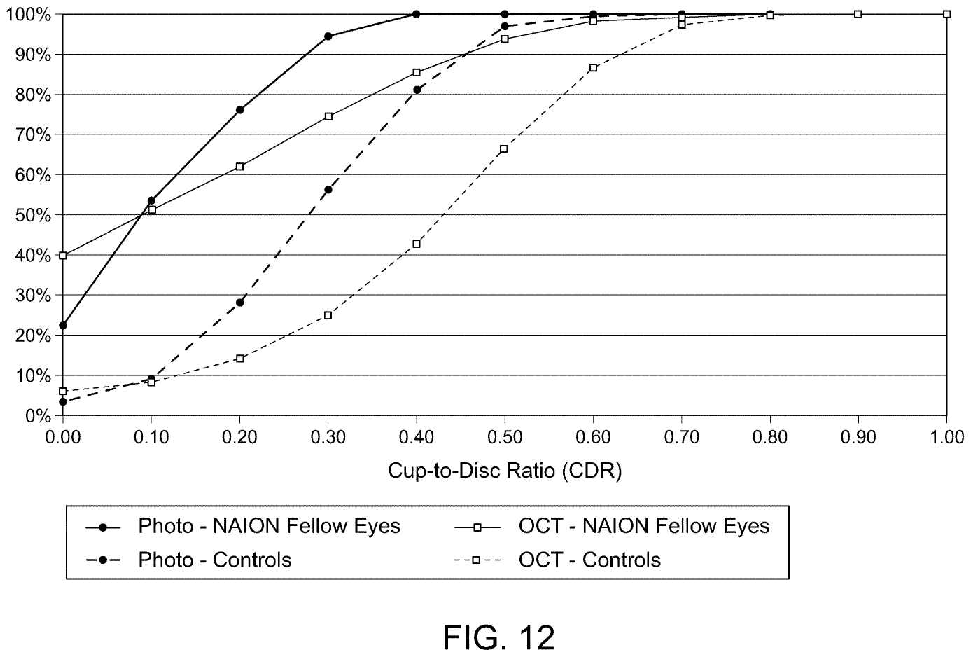

FIG. 12 shows the cumulative distribution of photo-measured and OCT-measured CDR among unaffected fellow eyes of NAION patients (all medication groups) compared with the photo-measured and OCT-measured CDR of the control group. The leftward shift of the fellow eyes (photo-measured and OCT-measured) is evidence of the smaller CDR in patients with NAION as compared with controls. The average photo-measured CDR in the fellow eye group (0.20) was significantly smaller than the average photo-measured CDR of the control group (0.37; p<0.001), and the average OCT-measured CDR in the fellow eye group (0.25) was significantly smaller than the average OCT-measured CDR of the control group (0.50; p<0.001).

Table 3 summarizes the odds of an eye having NAION based on its CDR. The table compares the number of unaffected fellow eyes of NAION patients in each CDR group to the number of eyes in the low-risk group (CDR>0.40). The odds ratio calculated in the table is the increased odds of having NAION in a certain CDR group as compared with eyes in the low-risk CDR group (>0.40). In the NAION patient group, 54% of the fellow eyes had a photo-measured CDR≤0.20, whereas only 9% of control eyes had a photo-measured CDR in this range. Eyes with a photo-measured CDR≤0.20 have 46.3 times higher odds of NAION than eyes with a CDR>0.40, and eyes with a CDR>0.20 and ≤0.40 have 6.8 times higher odds of NAION than eyes with a CDR>0.40.

TABLE 3
Odds ratio for CDR groups.

| | | | | |
|---|---|---|---|---|
| | NAION | | Odds | 95% Confidence |
| CDR Group | Fellow Eyes | Control Eyes | Ratio | Interval |

Photo-measured CDR

| | | | | |
|---|---|---|---|---|
| ≤0.20 | 126 (54%) | 85 (9%) | 46.3 | [22.9-93.5] |
| >0.20 & ≤0.30 | 53 (23%) | 177 (19%) | 9.4 | [5.0-17.6] |
| >0.30 & ≤0.40 | 43 (18%) | 262 (28%) | 5.1 | [2.7-9.7] |
| >0.40 & <0.50 | 13 (6%) | 230 (5%) | 1.8 | [0.8-3.9] |
| High Risk (≤0.20) | 126 (54%) | 85 (9%) | 46.3 | [22.9-93.5] |
| Moderate Risk (>0.20 | 96 (41%) | 439 (47%) | 6.8 | [3.5-13.2] |
| & <0.40) | | | | |
| Low Risk (>0.40) | 13 (6%) | 406 (44%) | 1.0 | N/A |

OCT-measured CDR

| | | | | |
|---|---|---|---|---|
| ≤0.20 | 335 (51%) | 77 (8%) | 18.2 | [13.5-24.5] |
| >0.20 & ≤0.30 | 71 (11%) | 55 (6%) | 5.4 | [3.7-8.0] |
| >0.30 & ≤0.40 | 81 (12%) | 100 (11%) | 3.1 | [2.4-4.8] |
| >0.40 & ≤0.50 | 72 (11%) | 166 (18%) | 1.8 | [1.3-2.5] |
| High Risk (≤0.20) | 335 (51%) | 77 (8%) | 18.2 | [13.5-24.5] |
| Moderate Risk (>0.20 | 152 (23%) | 155 (17%) | 4.1 | [3.1-5.4] |
| & <0.40) | | | | |
| Low Risk (>0.40) | 167 (26%) | 698 (75%) | 1.0 | N/A |

The primary goal of our study was to determine whether the optic discs of fellow eyes of patients with NAION after exposure to GLP-1 RA drugs have the same high prevalence of structurally crowded discs as fellow eyes of NAION patients with no GLP-1 RA exposure. The results confirm that the optic discs in these eyes are virtually identical with respect to CDR (Table 2 and FIG. 11). These observations are consistent with our hypothesis that GLP-1 RA associated NAION is not a distinct clinical entity, at least with respect to optic disc characteristics.

A secondary goal of this study was to create a data-driven assessment of risk in relation to CDR. Previous studies have identified a high prevalence of structurally crowded discs in patients with NAION, but none have stratified risk in relation to CDR. The images of 930 control eyes were analyzed using the same CDR measurement techniques that were used to assess the photographs from NAION patients. The results here demonstrate that the risk associated with CDR is not binary; the risk is inversely proportional to the CDR, at least for eyes with a CDR≤0.40 (Table 3). Specifically, eyes with a very small, photo-measured CDR (≤0.20) have 46 times greater odds of having NAION than eyes with a CDR>0.40, whereas eyes with a CDR>0.20 and ≤0.40 have only 7 times greater odds of having NAION than eyes with a CDR>0.40. Given this significant difference, we propose classifying eyes with a photo-measured CDR≤0.20 as high-risk for NAION, those with a photo-measured CDR>0.20 and ≤0.40 as moderate-risk, and those with a photo-measured CDR>0.40 as low-risk.

The results suggests the risk of NAION is not evenly distributed throughout the population. The comparisons of NAION patients with a control group designed to represent the general population suggests that 54% of all NAION occurs in the 9% of eyes with high-risk discs, and 95% occurs in the 56% of eyes with moderate or high-risk discs. The risk of NAION further increases if the patient is male and may more than double with the use of semaglutide. Although the GLP-1 RA associated risk is greatest 6 to 12 months after initiation of therapy, the medication induced risk continues for at least 4 years.

Our data demonstrates an increased prevalence of bilateral (likely non-simultaneous sequential) NAION in patients on semaglutide (29.9%) compared with NAION patients with no history of GLP-1 RA use (15.5%) (p<0.001). NAION also occurs at a younger age in patients on semaglutide (60.6 vs 62.8 years, p=0.005). To our knowledge, these statistically significant relationships have not been reported previously and may serve as additional evidence that semaglutide is associated with a greater risk of NAION.

Our study aligns with multiple other studies that identified a small CDR as the single most significant risk factor for NAION, but other risk factors also play a role. In our study, 97% of all NAION patients were over 40 years of age, and men were more likely to have NAION than women (59% vs 41%, p<0.001). We also noted that the 37% prevalence of diabetes in our NAION patients was much higher than the reported 19% prevalence of diabetes in the US population of adults over 45 of age.28 These data are consistent with other studies of NAION risk factors, although the risk related to diabetes has not been demonstrated consistently.

The study compared the estimated CDR for each eye as reported by the examining neuro-ophthalmologist with the measured CDR based on our annotations on the fundus photographs. The photo-measured CDR is more objective than physician estimations in that the annotations enable mathematical calculation of the areas of the labeled ellipses. Notably, the physicians' estimations of CDR averaged 35% smaller than the objective measurements. This is why 95% of NAION fellow eyes have a provider-estimated CDR≤0.30, but only 76% have a photo-measured CDR 0.30. Clinicians must be aware of this difference in measurement techniques when making clinical decisions about the risk of NAION for an individual patient.

The average photo-measured CDR of the control eyes in our study was 24% smaller than when measured with OCT (Topcon Maestro). This difference between OCT and photographic measurements is consistent with other reports and is not surprising because the anatomical landmarks assessed are not necessarily the same. Nevertheless, the data demonstrate good correlation between OCT and photographic measurements after accounting for this systematic quantitative difference.

The same high prevalence of structurally crowded discs in GLP-1 RA associated NAION as in NAION patients without GLP-1 RA exposure was observed. This data demonstrate that an eye's odds of NAION are inversely proportional to the CDR: the smaller the CDR, the greater the odds. Although NAION is a relatively rare phenomenon in the general population, eyes with high-risk optic discs (9% of the population) have 46 times greater odds of having an NAION event than eyes with low-risk discs. For this reason, it may be appropriate to screen patients for at-risk optic discs as part of the informed consent process before prescribing or continuing medications associated with a higher incidence of NAION.

From the foregoing, it will be appreciated that the present disclosure can be embodied in various ways, which include but are not limited to the following:

### LIST OF EMBODIMENTS

Embodiment 1. A method for determining a disc-at-risk in a given eye of a given patient, the method comprising the steps:
a. training a machine learning model with a plurality of eye fundus images from a plurality of patients;
b. inputting an image of the given eye of the patient into the machine learning model; and
c. outputting a status of disk-at-risk for the given patient and a corresponding confidence level.

Embodiment 2. The method of Embodiment 1, wherein the status of disc-at-risk is binary or ternary.

Embodiment 3. The method of any one of Embodiments 1-2, wherein each of the plurality of eye fundus images from the plurality of patients further comprises an associated status of disc-at-risk for each image.

Embodiment 4. A computer-implemented method for analyzing an image of a fundus of an eye for determining whether the eye has a disc-at-risk, the method comprising the steps:
a. receiving the image of a fundus of the eye;
b. detecting, using a first segmentation model, a perimeter of a cup;
c. detecting, using a second segmentation model, a perimeter of a disc;
d. calculating a physical characteristic of the optic disc from the detected perimeter of the cup and the detected perimeter of the disc;
e. determining, based on the calculated physical characteristic of the optic disc and the image of the fundus as inputs, that the eye has a disc-at-risk; and
f. providing a notification that the eye has a disc-at-risk.

Embodiment 5. The method of Embodiment 4, wherein the physical characteristic of the optic disc is a ratio of the surface areas of the cup and the optic disc.

Embodiment 6. The method of Embodiment 4, wherein the physical characteristic of the optic disc is a ratio of the diameters of the cup and the optic disc.

Embodiment 7. The method of any one of Embodiments 4-6, wherein the first segmentation model is trained using a plurality of images of fundus where the perimeter of the cup in each image has been determined.

Embodiment 8. The method of any one of Embodiments 4-7, wherein the second segmentation model is trained using a plurality of images of fundus where the perimeter of the disc in each image has been determined.

Embodiment 9. The method of any one of Embodiments 4-8, wherein the first and second segmentation models are convolutional neural networks.

Embodiment 10. A computer-implemented method for analyzing an image of a fundus for determining whether an eye has a disc-at-risk, the method comprising the steps:
a. receiving an image of a fundus of an eye;
b. determining, using a first AI classification model, (i) a first status of whether the eye has a disc with minimal risk, and (ii) a first corresponding confidence level;
c. determining, using a second AI classification model, (i) a second status of whether the eye has a disc with some risk, and (ii) a second corresponding confidence level; and
d. determining, using a third AI classification model, (i) a third status of whether the eye has a disc with high risk, and (ii) a third corresponding confidence level.

Embodiment 11. The method of Embodiment 10, further comprising the steps;
a. inputting, into an ensemble model, the first status of whether the eye has a disc with minimal risk, the first corresponding confidence level, the second status of whether the eye has a disc with some risk, the second corresponding confidence level, the third status of whether the eye has a disc with high risk, and the third corresponding confidence level;
b. determining, using the ensemble model, (i) a fourth status of whether the eye has a disc-at-risk, and (ii) a fourth corresponding confidence level; and
c. providing a notification that the eye has a disc-at-risk based on the fourth status.

Embodiment 12. The method of Embodiment 10 or 11, wherein the first status of whether the eye has a disc with minimal risk, the second status of whether the eye has a disc with some risk, and the third status of whether the eye has a disc with high risk are binary.

Embodiment 13. The method of any one of Embodiments 10-12, wherein the first AI classification model is trained with a plurality of images of fundus, wherein each image in the plurality of images has been assigned a status of whether the eye has a disc with minimal risk.

Embodiment 14. The method of any one of Embodiments 10-13, wherein the second AI classification mode is trained with a plurality of images of fundus, wherein each image in the plurality of images has been assigned a status of whether the eye has a disc with some risk.

Embodiment 15. The method of any one of Embodiments 10-14, wherein the third AI classification model is trained with a plurality of images of fundus, wherein each image in the plurality of images has been assigned a status of whether the eye has a disc with high risk.

Embodiment 16. A computer-implemented method for analyzing an image of a fundus for determining whether an eye has a disc-at-risk, the method comprising the steps:
a. receiving an image of a fundus of an eye; and
b. determining, using an AI classification model, a risk status of the eye and a corresponding confidence level, wherein the risk status of the eye is selected from the group consisting of a disc with minimal risk, a disc with some risk, and a disc with high risk.

Embodiment 17. The method of Embodiment 16, wherein the AI classification model is trained with a plurality of images of fundus, wherein each image in the plurality of images has been assigned a status of whether the eye has a disc with minimal risk a disc with some risk, or a disc with high risk.

Embodiment 18. A computer-implemented method of determining an increased risk of developing NAION in an eye of a patient, the method comprising the steps:
a. receiving the image of a fundus of the eye;
b. detecting, using a first segmentation model, a perimeter of a cup;
c. detecting, using a second segmentation model, a perimeter of a disc;
d. calculating a physical characteristic of the optic disc from the detected perimeter of the cup and the detected perimeter of the disc;
e. determining, based on the calculated physical characteristic of the optic disc and the image of the fundus as inputs, that the eye has an increased risk of developing NAION; and
f. providing a notification that the eye has an increased risk of developing NAION.

Embodiment 19. The method of Embodiment 18, wherein the calculated physical characteristic of the optic disc is the C/D ratio.

Embodiment 20. The method of Embodiment 19, wherein the eye is determined to have an increased risk of developing NAION when the CDR is less than or equal to about 0.2.

Embodiment 21. A computer-implemented method for determining an increased risk of developing NAION in an eye of a patient, the method comprising the steps:
a. receiving an image of a fundus of the eye;
b. determining, using a first AI classification model, (i) a first status of whether the eye has a disc with minimal risk, and (ii) a first corresponding confidence level;
c. determining, using a second AI classification model, (i) a second status of whether the eye has a disc with some risk, and (ii) a second corresponding confidence level;
d. determining, using a third AI classification model, (i) a third status of whether the eye has a disc with high risk, and (ii) a third corresponding confidence level;
e. inputting, into an ensemble model, the first status of whether the eye has a disc with minimal risk, the first corresponding confidence level, the second status of whether the eye has a disc with some risk, the second corresponding confidence level, the third status of whether the eye has a disc with high risk, and the third corresponding confidence level;
f. determining, using the ensemble model, (i) a fourth status of whether the eye has a disc-at-risk, and (ii) a fourth corresponding confidence level; and
g. providing a notification that the eye has an increased risk of developing NAION based on the fourth status.

Embodiment 22. The method of Embodiment 21, wherein the first status of whether the eye has a disc with minimal risk, the second status of whether the eye has a disc with some risk, and the third status of whether the eye has a disc with high risk are binary.

Embodiment 23. The method of Embodiment 21 or 22, wherein the first AI classification model is trained with a plurality of images of fundus, wherein each image in the plurality of images has been assigned a status of whether the eye has a disc with minimal risk.

Embodiment 24. The method of any one of Embodiments 21-23, wherein the second AI classification mode is trained with a plurality of images of fundus, wherein each image in the plurality of images has been assigned a status of whether the eye has a disc with some risk.

Embodiment 25. The method of any one of Embodiments 21-24, wherein the third AI classification model is trained with a plurality of images of fundus, wherein each image in the plurality of images has been assigned a status of whether the eye has a disc with high risk.

Embodiment 26. The method of any one of Embodiments 21-25, wherein the C/D ratio of an eye having a disc-at-risk is less than or equal to about 0.2.

Embodiment 27. A computer-implemented method for determining whether an eye has an increased risk of developing NAION, the method comprising the steps:
a. receiving an image of a fundus of an eye;
b. determining, using an AI classification model, a risk status of the eye and a corresponding confidence level, wherein the risk status of the eye is selected from the group consisting of a disc with minimal risk, a disc with some risk, and a disc with high risk; and
c. providing a notification that the eye has an increased risk of developing NAION when the risk status of the eye is a disc with high risk.

Embodiment 28. The method of Embodiment 27, wherein the AI classification model is trained with a plurality of images of fundus, wherein each image in the plurality of images has been assigned a status of whether the eye has a disc with minimal risk a disc with some risk, or a disc with high risk.

Embodiment 29. The method of Embodiment 28, wherein the C/D ratio of the eye with a risk status of a disc with high risks less than or equal to about 0.2.

Embodiment 30. A program which, when executed by a processor, causes the processor to perform a method according to any one of Embodiments 1-29.

Embodiment 31. A computer-readable storage medium storing a program according to Embodiment 18.

Embodiment 32. A non-transitory computer-readable medium storing instructions that, when executed by a processor, causes the processor to perform the method of any one of Embodiments 1-29.

Embodiment 33. A non-transitory computer-readable storage medium comprising a report generated from performing the method of any one of Embodiments 1-29.

Embodiment 34. An electronic device, comprising: one or more processors; a memory storing instructions that, when executed by the one or more processors, cause the one or more processors to perform the method of any one of Embodiments 1-29.

Embodiment 35. The electronic device of Embodiment 34, further comprising one or more displays to present a report generated from performing the method of any one of Embodiments 1-29.

Embodiment 36. A system for analyzing an image of a fundus of an eye for determining whether the eye has a disc-at-risk, the system comprising:
a. a camera capable of taking the image of the fundus of the eye;
b. a processor communicatively coupled to the camera; and
c. a non-transitory computer readable media communicatively coupled to the processor, and storing instructions that, when executed on the processor, causes the processor to perform the steps of:
   i. detecting, using a first segmentation model, a perimeter of a cup, wherein the first segmentation model is trained on cup segmentation data;
   ii. detecting, using a second segmentation model, a perimeter of a disc;
   iii. calculating a physical characteristic of the fundus from the detected perimeter of the cup and the detected perimeter of the disc;
   iv. determining, based on the calculated physical characteristic of the optic disc and the image of the fundus as inputs, that the eye has a disc-at risk; and
   v. providing a notification, via an output device, that the eye has a disc-at-risk.

Embodiment 37. The system of Embodiment 36, wherein the physical characteristic of the optic disc is a ratio of the surface areas of the cup and the optic disc.

Embodiment 38. The system of Embodiment 36, wherein the physical characteristic of the optic disc is a ratio of the diameters of the cup and the optic disc.

Embodiment 39. The system of any one of Embodiments 36-38, wherein the first segmentation model is trained using a plurality of images of fundus where the perimeter of the cup in each image has been determined.

Embodiment 40. The system of any one of Embodiments 36-39, wherein the second segmentation model is trained using a plurality of images of fundus where the perimeter of the disc in each image has been determined.

Embodiment 41. A system for analyzing an image of a fundus of an eye for determining whether the eye has a disc-at-risk, the system comprising:
a. a camera capable of taking the image of the fundus of the eye;
b. a processor communicatively coupled to the camera; and
c. a non-transitory computer readable media communicatively coupled to the processor, and storing instructions that, when executed on the processor, causes the processor to perform the steps of
   i. receiving an image of a fundus of an eye;
   ii. determining, using a first AI classification model, (i) a first status of whether the eye has a disc with minimal risk, and (ii) a first corresponding confidence level;
   iii. determining, using a second AI classification model, (i) a second status of whether the eye has a disc with some risk, and (ii) a second corresponding confidence level;
   iv. determining, using a third AI classification model, (i) a third status of whether the eye has a disc with high risk, and (ii) a third corresponding confidence level; and
   v. providing a notification, via an output device, that the eye has a disc-at-risk if the third status of whether the eye has a disc with high risk is true.

Embodiment 42. The system of Embodiment 41, wherein the non-transitory computer readable media communicatively coupled to the processor, and storing instructions that, when executed on the processor, causes the processor to perform the further steps of:
i. inputting, into an ensemble model, the first status of whether the eye has a disc with minimal risk, the first corresponding confidence level, the second status of whether the eye has a disc with some risk, the second corresponding confidence level, the third status of whether the eye has a disc with high risk, and the third corresponding confidence level;
   ii. determining, using the ensemble model, (i) a fourth status of whether the eye has a disc-at-risk, and (ii) a fourth corresponding confidence level; and
   iii. providing a notification that the eye has a disc-at-risk based on the fourth status.

Embodiment 43. The system of Embodiment 41 or 42, wherein the first status of whether the eye has a disc with minimal risk, the second status of whether the eye has a disc with some risk, and the third status of whether the eye has a disc with high risk are binary.

Embodiment 44. The system of any one of Embodiments 41-43, wherein the first AI classification model is trained with a plurality of images of fundus, wherein each image in the plurality of images has been assigned a status of whether the eye has a disc with minimal risk.

Embodiment 45. The system of any one of Embodiments 41-44, wherein the second AI classification mode is trained with a plurality of images of fundus, wherein each image in the plurality of images has been assigned a status of whether the eye has a disc with some risk.

Embodiment 46. The system of any one of Embodiments 41-45, wherein the third AI classification model is trained with a plurality of images of fundus, wherein each image in the plurality of images has been assigned a status of whether the eye has a disc with high risk.

Embodiment 47. A system for analyzing an image of a fundus of an eye for determining whether the eye has a disc-at-risk, the system comprising:
a. a camera capable of taking an image of the fundus of the eye;
b. a processor communicatively coupled to the camera; and
c. a non-transitory computer readable media communicatively coupled to the processor, and storing instructions that, when executed on the processor, causes the processor to perform the steps of:
   i. receiving an image of a fundus of an eye;
   ii. determining, using an AI classification model, a risk status of the eye and a corresponding confidence level, wherein the risk status of the eye is selected from the group consisting of a disc with minimal risk, a disc with some risk, and a disc with high risk; and
   iii. providing a notification that the eye has a disc-at-risk when the risk status of the eye is a disc with high risk.

Embodiment 48. The system of Embodiment 47, wherein the AI classification model is trained with a plurality of images of fundus, wherein each image in the plurality of images has been assigned a status of whether the eye has a disc with minimal risk a disc with some risk, or a disc with high risk.

Embodiment 49. A system for analyzing an image of a fundus of an eye for determining whether the eye has an increased risk of developing NAION, the system comprising:
a. a camera capable of taking the image of the fundus of the eye;
b. a processor communicatively coupled to the camera; and
c. a non-transitory computer readable media communicatively coupled to the processor, and storing instructions that, when executed on the processor, causes the processor to perform the steps of:
   i. detecting, using a first segmentation model, a perimeter of a cup, wherein the first segmentation model is trained on cup segmentation data;
   ii. detecting, using a second segmentation model, a perimeter of a disc;
   iii. calculating a physical characteristic of the fundus from the detected perimeter of the cup and the detected perimeter of the disc;
   iv. determining, based on the calculated physical characteristic of the optic disc and the image of the fundus as inputs, that the eye has an increased risk of developing NAION; and
   v. providing a notification, via an output device, that the eye has an increased risk of developing NAION.

Embodiment 50. The system of Embodiment 49, wherein the physical characteristic of the optic disc is a ratio of the surface areas of the cup and the optic disc.

Embodiment 51. The system of Embodiment 49, wherein the physical characteristic of the optic disc is a ratio of the diameters of the cup and the optic disc.

Embodiment 52. The system of any one of Embodiments 49-51, wherein the first segmentation model is trained using a plurality of images of fundus where the perimeter of the cup in each image has been determined.

Embodiment 53. The system of any one of Embodiments 49-52, wherein the second segmentation model is trained using a plurality of images of fundus where the perimeter of the disc in each image has been determined.

Embodiment 54. A system for analyzing an image of a fundus of an eye for determining whether the eye has an increased risk of developing NAION, the system comprising:
a. a camera capable of taking the image of the fundus of the eye;
b. a processor communicatively coupled to the camera; and
c. a non-transitory computer readable media communicatively coupled to the processor, and storing instructions that, when executed on the processor, causes the processor to perform the steps of:
   i. receiving an image of a fundus of an eye;
   ii. determining, using a first AI classification model, (i) a first status of whether the eye has a disc with minimal risk, and (ii) a first corresponding confidence level;
   iii. determining, using a second AI classification model, (i) a second status of whether the eye has a disc with some risk, and (ii) a second corresponding confidence level;
   iv. determining, using a third AI classification model, (i) a third status of whether the eye has a disc with high risk, and (ii) a third corresponding confidence level;
   v. determining, using the ensemble model, (i) a fourth status of whether the eye has a disc-at-risk, and (ii) a fourth corresponding confidence level, and
   vi. providing a notification, via an output device, that the eye has an increased risk of developing NAION based on the fourth status.

Embodiment 55. The system of Embodiment 54, wherein the first status of whether the eye has a disc with minimal risk, the second status of whether the eye has a disc with some risk, and the third status of whether the eye has a disc with high risk are binary.

Embodiment 56. The system of Embodiment 54 or 55, wherein the first AI classification model is trained with a plurality of images of fundus, wherein each image in the plurality of images has been assigned a status of whether the eye has a disc with minimal risk.

Embodiment 57. The system of any one of Embodiments 54-56, wherein the second AI classification mode is trained with a plurality of images of fundus, wherein each image in the plurality of images has been assigned a status of whether the eye has a disc with some risk.

Embodiment 58. The system of any one of Embodiments 54-57, wherein the third AI classification model is trained with a plurality of images of fundus, wherein each image in the plurality of images has been assigned a status of whether the eye has a disc with high risk.

Embodiment 59. A system for analyzing an image of a fundus of an eye for determining whether the eye has an increased risk of developing NAION, the system comprising:
a. a camera capable of taking an image of the fundus of the eye;
b. a processor communicatively coupled to the camera; and
c. a non-transitory computer readable media communicatively coupled to the processor, and storing instructions that, when executed on the processor, causes the processor to perform the steps of.
   i. receiving an image of a fundus of an eye;
   ii. determining, using an AI classification model, a risk status of the eye and a corresponding confidence level, wherein the risk status of the eye is selected from the group consisting of a disc with minimal risk, a disc with some risk, and a disc with high risk; and
   iii. providing a notification that the eye has an increased risk of developing NAION when the risk status of the eye is a disc with high risk.

Embodiment 60. The system of Embodiment 59, wherein the AI classification model is trained with a plurality of images of fundus, wherein each image in the plurality of images has been assigned a status of whether the eye has a disc with minimal risk a disc with some risk, or a disc with high risk.

Embodiment 61. A method for determining a disc-at-risk in a given eye of a given patient, the method comprising the steps:
a. Training a machine learning model with a plurality of eye fundus images from a plurality of patients;
b. Receiving an image of the given eye of the patient; and
c. Outputting a status of disk-at-risk for the given patient.

Embodiment 62. The method of Embodiment 61, wherein the status of disc-at-risk is comprises a binary value.

Embodiment 63. The method of any one of Embodiments 61-62, wherein each of the plurality of eye fundus images from the plurality of patients further comprises an associated status of disc-at-risk for each image.

Embodiment 64. A method of determining whether an eye has a disc-at-risk, the method comprising the steps:
a. Receiving an image of a fundus of an eye;
b. Detecting, using a first AI model, a perimeter of a cup, wherein the first AI model is trained on cup segmentation data;
c. Detecting, using a second AI model, a perimeter of a disc, wherein the second AI model is trained on disc segmentation data;
d. Calculating a physical characteristic of the fundus from the detected perimeter of the cup and the detected perimeter of the disc;
e. Determining, using an AI classifier model based on the calculated physical characteristic of the fundus and the image of the fundus as inputs, that the eye has a disc-at-risk; and
f. Providing a notification that the eye has a disc-at-risk.

Embodiment 65. The method of Embodiment 64, wherein the physical characteristic of the fundus is a ratio of the surface areas of the cup and the disc.

Embodiment 66. A method of determining whether an eye has a disc-at-risk, the method comprising the steps:
a. Receiving an image of a fundus of an eye;
b. Detecting, using an AI model, a perimeter of a disc, wherein the AI model is trained on disc segmentation data;
c. Determining, using an AI classifier model based on the detected perimeter of the disc and the image of the fundus as inputs, that the eye has a disc-at-risk; and
d. Providing a notification that the eye has a disc-at-risk.

Embodiment 67. A method of determining whether an eye has a disc-at-risk, the method comprising the steps:
a. Receiving an image of a fundus of an eye;
b. Detecting, using an AI model, a perimeter of a disc, wherein the AI model is trained on disc segmentation data;
c. Calculating the distribution of colors detected by the pixels within the detected perimeter of the disc;
d. Determining, using an AI classifier model based on the distribution of colors detected by the pixels within the detected perimeter of the disc, that the eye has a disc-at risk; and
e. Providing a notification that the eye has a disc-at-risk.

Embodiment 68. A method of determining whether an eye has a disc-at-risk from an image of a fundus of the eye, the method comprising the steps:
a. Identifying the eye as having a disc-at-risk using an AI classifier model, wherein the AI classifier model is based on one or more risk prediction values and one or more corresponding confidence levels generated by one or more AI risk prediction models selected from the group consisting of a C/D Model, a DAR Model, and a Color Model,
   i. wherein generating a risk prediction value and a corresponding confidence level using the C/D Model comprises the steps:
      1. Receiving an image of a fundus of an eye;
      2. Detecting, using a first AI model, a perimeter of a cup, wherein the first AI model is trained on cup segmentation data;
      3. Detecting, using a second AI model, a perimeter of a disc, wherein the second AI model is trained on disc segmentation data;
      4. Calculating a physical characteristic of the fundus from the detected perimeter of the cup and the detected perimeter of the disc; and
      5. Generating the risk prediction value and the corresponding confidence level that the eye has a disc-at risk, using an AI classifier model based on the calculated physical characteristic of the fundus and the image of the fundus as inputs;
   ii. wherein generating a risk prediction value and a corresponding confidence level using the DAR Model comprises the steps:
      1. Receiving the image of a fundus of an eye;
      2. Detecting, using a third AI model, a perimeter of a disc, wherein the third AI model is trained on disc segmentation data; and
      3. Generating the risk prediction value and the corresponding confidence level that the eye has a disc-at risk, using an AI classifier model based on the detected perimeter of the disc and the image of the fundus as inputs;
   iii. And wherein generating a risk prediction value and a corresponding confidence level using the Color Model comprises the steps:
      1. Receiving the image of a fundus of an eye;
      2. Detecting, using a fourth AI model, to detect a perimeter of a disc, wherein the fourth AI model is trained on disc segmentation data; and
      3. Generating the risk prediction value and the corresponding confidence level that the eye has a disc-at risk, using an AI classifier model based on the distribution of colors detected by the pixels within the detected perimeter of the disc and the image of the fundus as inputs.

Embodiment 69. The method of Embodiment 68, wherein the physical characteristic of the fundus is a ratio of the surface areas of the cup and the disc.

Embodiment 70. The method of Embodiment 68 or 69, wherein an overall risk prediction value and an overall confidence level that the eye has a disc-at-risk is calculated from the one or more risk prediction values generated by the one or more AI risk prediction models selected from the group consisting of a C/D Model, a DAR Model, and a Color Model, weighted by the one or more corresponding confidence levels.

Embodiment 71. A method of determining an increased risk of developing NAION in an eye of a patient, the method comprising the steps:
a. Receiving an image of a fundus of the eye of the patient;
b. Detecting, using an AI model, a perimeter of a disc, wherein the AI model is trained on disc segmentation data;
c. Calculating, using an AI classifier model, a risk prediction value and a confidence level that the eye has a disc-at-risk, wherein the AI classifier model is based on the image of the fundus and the perimeter of the disc as inputs;
d. Determining that the patient has an increased risk of developing NAION when the risk prediction value is greater than a first predetermined threshold; and
e. Determining that the patient has no risk of developing NAION when the risk prediction value is less than the first predetermined threshold.

Embodiment 72. The method of Embodiment 71, wherein the risk prediction value is 0 or 1.

Embodiment 73. The method of Embodiment 71, wherein the AI classifier model is tuned such that a predetermined fraction of all patients have a risk prediction value of 1.

Embodiment 74. A method of determining an increased risk of developing NAION in an eye of a patient, the method comprising the steps:
a. Receiving an image of a fundus of the eye of the patient;
b. Detecting, using a first AI model, a perimeter of a cup, wherein the first AI model is trained on cup segmentation data;
c. Detecting, using a second AI model, a perimeter of a disc, wherein the second AI model is trained on disc segmentation data;
d. Calculating a risk prediction value and a confidence level that the eye has a disc-at-risk using an AI classifier model, wherein the AI classifier model is based on the image of the fundus, the perimeter of the cup, and the perimeter of the disc as inputs;
e. Determining that the patient has an increased risk of developing NAION when the risk prediction value is greater than a first predetermined threshold; and
f. Determining that the patient has no risk of developing NAION when the risk prediction value is less than the first predetermined threshold.

Embodiment 75. The method of Embodiment 74, wherein the risk prediction value is 0 or 1.

Embodiment 76. The method of Embodiment 74, wherein the AI classifier model is tuned such that a predetermined fraction of all patients have a risk prediction value of 1.

Embodiment 77. A method of determining an increased risk of developing NAION in an eye of a patient, the method comprising the steps:
a. Receiving an image of a fundus of the eye of the patient;
b. Calculating a risk prediction value and a confidence level that the eye has a disc-at-risk using an AI classifier model, wherein the AI classifier model is based on the image of the fundus as input;
c. Determining that the patient has an increased risk of developing NAION when the risk prediction value is greater than a first predetermined threshold; and
d. Determining that the patient has no risk of developing NAION when the risk prediction value is less than the first predetermined threshold.

Embodiment 78. The method of Embodiment 77, wherein the risk prediction value is 0 or 1.

Embodiment 79. The method of Embodiment 77, wherein the AI classifier model is tuned such that a predetermined fraction of all patients have a risk prediction value of 1.

Embodiment 80. A method of determining an increased risk of developing NAION in an eye of a patient, the method comprising the steps:
a. Receiving an image of a fundus of the eye of the patient;
b. Detecting, using an AI model, a perimeter of a disc, wherein the AI model is trained on disc segmentation data;
c. Calculating a color distribution of pixels in the perimeter of the disc;
d. Calculating a risk prediction value and a confidence level that the eye has a disc-at-risk using an AI classifier model, wherein the AI classifier model is based on the color distribution of pixels in the perimeter of the disc;
e. Determining that the patient has an increased risk of developing NAION when the risk prediction value is greater than a first predetermined threshold; and
f. Determining that the patient has no risk of developing NAION when the risk prediction value is less than the first predetermined threshold.

Embodiment 81. The method of Embodiment 80, wherein the risk prediction value is 0 or 1.

Embodiment 82. The method of Embodiment 80, wherein the AI classifier model is tuned such that a predetermined fraction of all patients have a risk prediction value of 1.

Embodiment 83. A method of determining an increased risk of developing NAION in an eye of a patient, the method comprising the steps:
a. Calculating a risk prediction value and a confidence level that the eye has a disc-at-risk using an AI classifier model, wherein the AI classifier model is based on a plurality of risk prediction values and a plurality of corresponding confidence levels generated by one or more AI risk prediction models selected from the group consisting of a C/D Model, a DAR Model, and a Color Model,
   i. wherein generating a risk prediction value and a corresponding confidence level using the C/D Model comprises the steps:
      1. Receiving the image of the fundus of the eye;
      2. Detecting, using a first AI model, a perimeter of a cup, wherein the first AI model is trained on cup segmentation data;
      3. Detecting, using a second AI model, a perimeter of a disc, wherein the second AI model is trained on disc segmentation data;
      4. Calculating a physical characteristic of the fundus from the detected perimeter of the cup and the detected perimeter of the disc; and
      5. Generating the risk prediction value and the corresponding confidence level that the eye has a disc-at risk, using an AI classifier model based on the calculated physical characteristic of the fundus and the image of the fundus as inputs;
   ii. wherein generating a risk prediction value and a corresponding confidence level using the DAR Model comprises the steps:
      1. Receiving the image of a fundus of an eye of the patient;
      2. Detecting, using a third AI model, a perimeter of a disc, wherein the third AI model is trained on disc segmentation data; and
      3. Generating the risk prediction value and the corresponding confidence level that the eye has a disc-at risk using an AI classifier model based on the detected perimeter of the disc and the image of the fundus as inputs;
   iii. wherein generating a risk prediction value and a corresponding confidence level using the Color Model comprises the steps:
      1. Receiving the image of a fundus of an eye of the patient;
      2. Detecting, using a fourth AI model, to detect a perimeter of a disc, wherein the fourth AI model is trained on disc segmentation data; and
   iv. Generating the risk prediction value and the corresponding confidence level that the eye has a disc-at risk, using an AI classifier model based on the distribution of colors detected by the pixels within the detected perimeter of the disc and the image of the fundus as inputs;
b. Determining that the patient has an increased risk of developing NAION when the risk prediction value is greater than a first predetermined threshold; and
c. Determining that the patient has no risk of developing NAION when the risk prediction value is less than the first predetermined threshold.

Embodiment 84. The method of Embodiment 83, wherein the risk prediction value is 0 or 1.

Embodiment 85. The method of Embodiment 83, wherein the AI classifier model is tuned such that a predetermined fraction of all patients have a risk prediction value of 1.

Embodiment 86. The method of Embodiment 83, wherein the risk prediction value and the confidence level that the eye has a disc-at-risk is calculated from the plurality of risk prediction values generated by the one or more AI risk prediction models selected from the group consisting of a C/D Model, a DAR Model, and a Color Model, weighted by the plurality of corresponding confidence levels.

Embodiment 87. The method of any one of Embodiments 71-86, wherein the method further comprises the steps:
a. Receiving one or more risk information about the patient, the risk information selected from the group consisting of age, gender, blood pressure, and health history;
b. Inputting the one or more risk information into the AI classifier model; and
c. Generating the risk prediction value and the corresponding confidence level that the eye has a disc-at risk using the AI classifier model, wherein the AI classifier model includes the one or more risk information as an input.

Embodiment 88. A non-transitory computer-readable medium storing instructions that, when executed by a processor, causes the processor to perform the method of any one of Embodiments 61-87.

Embodiment 89. A non-transitory computer-readable storage medium comprising a report generated from performing the method of any one of Embodiments 61-87.

Embodiment 90. An electronic device, comprising: one or more processors; a memory storing instructions that, when executed by the one or more processors, cause the one or more processors to perform the method of any one of Embodiments 61-87.

Embodiment 91. The electronic device of Embodiment 90, further comprising one or more displays to present a report generated from performing the method of any one of Embodiments 61-87.

Embodiment 92. A system for determining whether an eye has a disc-at-risk from an image of a fundus of the eye, the system comprising:
a. A camera capable of taking an image of the fundus of the eye;
b. A processor communicatively coupled to the camera; and
c. A non-transitory computer readable media communicatively coupled to the processor, and storing instructions that, when executed on the processor, causes the processor to perform the steps of:
   i. Detecting, using a first AI model, a perimeter of a cup, wherein the first AI model is trained on cup segmentation data;
   ii. Detecting, using a second AI model, a perimeter of a disc, wherein the second AI model is trained on disc segmentation data;
   iii. Calculating a physical characteristic of the fundus from the detected perimeter of the cup and the detected perimeter of the disc;
   iv. Determining, using an AI classifier model based on the calculated physical characteristic of the fundus and the image of the fundus as inputs, that the eye has a disc-at risk; and
   v. Providing a notification, via an output device, that the eye has a disc-at-risk.

Embodiment 93. A system for determining whether an eye has a disc-at-risk from an image of a fundus of the eye, the system comprising:
a. A camera capable of taking an image of the fundus of the eye;
b. A processor communicatively coupled to the camera; and
c. A non-transitory computer readable media communicatively coupled to the processor, and storing instructions that, when executed on the processor, causes the processor to perform the steps of:
   i. Detecting, using an AI model, a perimeter of a cup, wherein the AI model is trained on cup segmentation data;
   ii. Determining that the eye has a disc-at risk using an AI classifier model based on the detected perimeter of the disc and the image of the fundus as inputs; and
   iii. Providing a notification, via an output device, that the eye has a disc-at-risk.

Embodiment 94. A system for determining whether an eye has a disc-at-risk from an image of a fundus of the eye, the system comprising:
a. A camera capable of taking an image of the fundus of the eye;
b. A processor communicatively coupled to the camera; and
c. A non-transitory computer readable media communicatively coupled to the processor, and storing instructions that, when executed on the processor, causes the processor to perform the steps of:
   i. Using an AI model trained on disc segmentation to detect a perimeter of a disc;
   ii. Calculating the distribution of colors detected by the pixels within the detected perimeter of the disc;
   iii. Determining that the eye has a disc-at risk using an AI classifier model based on the distribution of colors detected by the pixels within the detected perimeter of the disc; and
   iv. Providing a notification, via an output device, that the eye has a disc-at-risk.

Embodiment 95. A system for determining an increased risk of developing NAION in an eye of a patient, the system comprising:
a. A camera capable of taking an image of the fundus of the eye;
b. A processor communicatively coupled to the camera; and
c. A non-transitory computer readable media communicatively coupled to the processor, and storing instructions that, when executed on the processor, causes the processor to perform the steps of:
   i. Detecting, using a first AI model, a perimeter of a cup, wherein the first AI model is trained on cup segmentation data;
   ii. Detecting, using a second AI model, a perimeter of a disc, wherein the second AI model is trained on disc segmentation data;
   iii. Calculating a physical characteristic of the fundus from the detected perimeter of the cup and the detected perimeter of the disc;
   iv. Calculating a risk prediction value and a confidence level, using an AI classifier model, wherein the AI classifier model is based on the calculated physical characteristic of the fundus and the image of the fundus as inputs;
   v. Providing a notification, via an output device, that the eye has an increased risk of developing NAION when the risk prediction value is greater than a first threshold and the confidence level is greater than a second threshold; and
   vi. Providing a notification, via an output device, that the eye has no risk of developing NAION when the risk prediction value is less than a third threshold and the confidence level is less than a fourth threshold.

Embodiment 96. A system for determining an increased risk of developing NAION in an eye of a patient, the system comprising:
a. A camera capable of taking an image of the fundus of the eye;
b. A processor communicatively coupled to the camera; and
c. A non-transitory computer readable media communicatively coupled to the processor, and storing instructions that, when executed on the processor, causes the processor to perform the steps of:
   i. Detecting, using an AI model, a perimeter of a disc, wherein the AI model is trained on disc segmentation data;
   ii. Calculating a risk prediction value and a confidence level, using an AI classifier model, wherein the AI classifier model is based on the perimeter of the disc and the image of the fundus as inputs;
   iii. Providing a notification, via an output device, that the eye has an increased risk of developing NAION when the risk prediction value is greater than a first threshold and the confidence level is greater than a second threshold; and
   iv. Providing a notification, via an output device, that the eye has no risk of developing NAION when the risk prediction value is less than a third threshold and the confidence level is less than a fourth threshold.

Embodiment 97. A system for determining an increased risk of developing NAION in an eye of a patient, the system comprising:
a. A camera capable of taking an image of the fundus of the eye;
b. A processor communicatively coupled to the camera; and
c. A non-transitory computer readable media communicatively coupled to the processor, and storing instructions that, when executed on the processor, causes the processor to perform the steps of:
   i. Detecting, using an AI model, a perimeter of a disc, wherein the AI model is trained on disc segmentation data;
   ii. Calculating the distribution of colors detected by the pixels within the detected perimeter of the disc;
   iii. Calculating a risk prediction value and a confidence level, using an AI classifier model, wherein the AI classifier model is based on the distribution of colors detected by the pixels within the detected perimeter of the disc and the image of the fundus as inputs;
   iv. Providing a notification, via an output device, that the eye has an increased risk of developing NAION when the risk prediction value is greater than a first threshold and the confidence level is greater than a second threshold; and
   v. Providing a notification, via an output device, that the eye has no risk of developing NAION when the risk prediction value is less than a third threshold and the confidence level is less than a fourth threshold.

Embodiment 98. A system for determining an increased risk of developing NAION in an eye of a patient, the system comprising:
a. A camera capable of taking an image of the fundus of the eye;
b. A processor communicatively coupled to the camera; and
c. A non-transitory computer readable media communicatively coupled to the processor, and storing instructions that, when executed on the processor, causes the processor to perform the steps of:
   i. Determining that the eye has an increased risk of developing NAION using an AI classifier model, wherein the AI classifier model is based on a plurality of risk prediction values and a plurality of corresponding confidence levels generated by one or more AI risk prediction models selected from the group consisting of a C/D Model, a DAR Model, and a Color Model,
      1. wherein generating a risk prediction value and a corresponding confidence level using the C/D Model comprises the steps:
         a. Receiving the image of the fundus of the eye;
         b. Using an AI model trained on cup segmentation to detect a perimeter of a cup;
         c. Using an AI model trained on disc segmentation to detect a perimeter of a disc;
         d. Calculating a physical characteristic of the fundus from the detected perimeter of the cup and the detected perimeter of the disc; and
         e. Generating the risk prediction value and the corresponding confidence level that the eye has a disc-at risk using an AI classifier model based on the calculated physical characteristic of the fundus and the image of the fundus as inputs;
      2. wherein generating a risk prediction value and a corresponding confidence level using the DAR Model comprises the steps:
         a. Receiving the image of a fundus of an eye of the patient;
         b. Using an AI model trained on disc segmentation to detect a perimeter of a disc; and
         c. Generating the risk prediction value and the corresponding confidence level, using an AI classifier model based on the detected perimeter of the disc and the image of the fundus as inputs;
      3. wherein generating a risk prediction value and a corresponding confidence level using the Color Model comprises the steps:
         a. Receiving the image of a fundus of an eye of the patient;
         b. Detecting, using an AI model trained on disc segmentation data, to detect a perimeter of a disc; and
         c. Generating the risk prediction value and the corresponding confidence level, using an AI classifier model, wherein the AI classifier model is based on the distribution of colors detected by the pixels within the detected perimeter of the disc and the image of the fundus as inputs;
   ii. Providing a notification, via an output device, that the eye has an increased risk of developing NAION when the risk prediction value is greater than a first threshold and the confidence level is greater than a second threshold; and
   iii. Providing a notification, via an output device, that the eye has no risk of developing NAION when the risk prediction value is less than a third threshold and the confidence level is less than a fourth threshold.

While the disclosure has been described with reference to particular aspects, it will be understood by those skilled in the art that various changes may be made and equivalents may be substituted for elements thereof without departing from the scope of the disclosure. In addition, many modifications may be made to a particular situation or material to the teachings of the disclosure without departing from the scope of the disclosure. Therefore, it is intended that the disclosure not be limited to the particular aspects disclosed but that the disclosure will include all aspects falling within the scope and spirit of the appended claims.

1.-9. (canceled)

10. A computer-implemented method for analyzing an image of a fundus for determining whether an eye has a disc-at-risk, the method comprising the steps:
a. receiving an image of a fundus of an eye;
b. determining, using a first AI classification model, (i) a first status of whether the eye has a disc with minimal risk, and (ii) a first corresponding confidence level;
c. determining, using a second AI classification model, (i) a second status of whether the eye has a disc with some risk, and (ii) a second corresponding confidence level; and
d. determining, using a third AI classification model, (i) a third status of whether the eye has a disc with high risk, and (ii) a third corresponding confidence level.

11. The method of claim 10, further comprising the steps:
a. inputting, into an ensemble model, the first status of whether the eye has a disc with minimal risk, the first corresponding confidence level, the second status of whether the eye has a disc with some risk, the second corresponding confidence level, the third status of whether the eye has a disc with high risk, and the third corresponding confidence level,
b. determining, using the ensemble model, (i) a fourth status of whether the eye has a disc-at-risk, and (ii) a fourth corresponding confidence level, and
c. providing a notification that the eye has a disc-at-risk based on the fourth status.

12. The method of claim 10, wherein the first status of whether the eye has a disc with minimal risk, the second status of whether the eye has a disc with some risk, and the third status of whether the eye has a disc with high risk are binary.

13. The method of claim 10, wherein the first AI classification model is trained with a plurality of images of fundus, wherein each image in the plurality of images has been assigned a status of whether the eye has a disc with minimal risk.

14. The method of claim 10, wherein the second AI classification mode is trained with a plurality of images of fundus, wherein each image in the plurality of images has been assigned a status of whether the eye has a disc with some risk.

15. The method of claim 10, wherein the third AI classification model is trained with a plurality of images of fundus, wherein each image in the plurality of images has been assigned a status of whether the eye has a disc with high risk.

16. A computer-implemented method for analyzing an image of a fundus for determining whether an eye has a disc-at-risk, the method comprising the steps:
a. receiving an image of a fundus of an eye;
b. determining, using an AI classification model, a risk status of the eye and a corresponding confidence level, wherein the risk status of the eye is selected from the group consisting of a disc with minimal risk, a disc with some risk, and a disc with high risk; and
c. providing a notification that the eye has a disc-at-risk when the risk status of the eye is a disc with high risk.

17. The method of claim 16, wherein the AI classification model is trained with a plurality of images of fundus, wherein each image in the plurality of images has been assigned a status of whether the eye has a disc with minimal risk, a disc with some risk, or a disc with high risk.

18.-20. (canceled)

21. A computer-implemented method for determining an increased risk of developing NAION in an eye of a patient, the method comprising the steps:
a. receiving an image of a fundus of the eye;
b. determining, using a first AI classification model, (i) a first status of whether the eye has a disc with minimal risk, and (ii) a first corresponding confidence level;
c. determining, using a second AI classification model, (i) a second status of whether the eye has a disc with some risk, and (ii) a second corresponding confidence level;
d. determining, using a third AI classification model, (i) a third status of whether the eye has a disc with high risk, and (ii) a third corresponding confidence level;
e. inputting, into an ensemble model, the first status of whether the eye has a disc with minimal risk, the first corresponding confidence level, the second status of whether the eye has a disc with some risk, the second corresponding confidence level, the third status of whether the eye has a disc with high risk, and the third corresponding confidence level,
f. determining, using the ensemble model, (i) a fourth status of whether the eye has an increased risk of developing NAION, and (ii) a fourth corresponding confidence level, and
g. providing a notification that the eye has an increased risk of developing NAION based on the fourth status.

22. The method of claim 21, wherein the first status of whether the eye has a disc with minimal risk, the second status of whether the eye has a disc with some risk, and the third status of whether the eye has a disc with high risk are binary.

23. The method of claim 21, wherein the first AI classification model is trained with a plurality of images of fundus, wherein each image in the plurality of images has been assigned a status of whether the eye has a disc with minimal risk.

24. The method of claim 21, wherein the second AI classification mode is trained with a plurality of images of fundus, wherein each image in the plurality of images has been assigned a status of whether the eye has a disc with some risk.

25. The method of claim 21, wherein the third AI classification model is trained with a plurality of images of fundus, wherein each image in the plurality of images has been assigned a status of whether the eye has a disc with high risk.

26. The method claim 21, wherein the C/D ratio of an eye having an increased risk of developing NAION is less than or equal to about 0.2.

27. A computer-implemented method for determining whether an eye has an increased risk of developing NAION, the method comprising the steps:
a. receiving an image of a fundus of an eye;
b. determining, using an AI classification model, a risk status of the eye and a corresponding confidence level, wherein the risk status of the eye is selected from the group consisting of a disc with minimal risk, a disc with some risk, and a disc with high risk; and
c. providing a notification that the eye has an increased risk of developing NAION when the risk status of the eye is a disc with high risk.

28. The method of claim 27, wherein the AI classification model is trained with a plurality of images of fundus, wherein each image in the plurality of images has been assigned a status of whether the eye has a disc with minimal risk, a disc with some risk, or a disc with high risk.

29. The method of claim 27, wherein the C/D ratio of the eye with a risk status of a disc with high risk is less than or equal to about 0.2.

30.-60. (canceled)

61. The method of claim 13, wherein the eye has a disc with minimal risk when the eye has a C/D ratio of greater than 0.4.

62. The method of claim 14, wherein the eye has a disc with some risk when the eye has a C/D ratio of greater than 0.2 and lesser than 0.4.

63. The method of claim 15, wherein the eye has a disc with high risk when the eye has a C/D ratio of lesser than or equal to 0.2.

64. The method of claim 11, wherein the eye has a disc-at-risk when the eye has a C/D ratio of lesser than or equal to 0.2.

65. The method of claim 17, wherein the risk status of the eye is a disc with minimal risk when the eye has a C/D ratio of greater than 0.4.

66. The method of claim 17, wherein the risk status of the eye is a disc with some risk when the eye has a C/D ratio of greater than 0.2 and lesser than 0.4.

67. The method of claim 17, wherein the risk status of the eye is a disc with high risk when the eye has a C/D ratio of lesser than 0.2.

68. The method of claim 23, wherein the eye has a disc with minimal risk when the eye has a C/D ratio of greater than 0.4.

69. The method of claim 24, wherein the eye has a disc with some risk when the eye has a C/D ratio of greater than 0.2 and lesser than 0.4.

70. The method of claim 25, wherein the eye has a disc with high risk when the eye has a C/D ratio of lesser than or equal to 0.2.

71. The method of claim 28, wherein the image in the plurality of images is assigned a status of whether the eye has a disc with minimal risk when the eye has a C/D ratio of greater than 0.4.

72. The method of claim 28, wherein the image in the plurality of images is assigned a status of whether the eye is a disc with some risk when the eye has a C/D ratio of greater than 0.2 and lesser than 0.4.

73. The method of claim 28, wherein the image in the plurality of images is assigned a status of whether the eye is a disc with high risk when the eye has a C/D ratio of lesser than 0.2.
